(19) United States
(12) Patent Application Publication
(10) Pub. No.: US 2025/0090312 A1
(43) Pub. Date: Mar. 20, 2025

(54) PROSTHETIC VALVES WITH INFORMATION INDICATORS

(71) Applicant: EDWARDS LIFESCIENCES CORPORATION, Irvine, CA (US)

(72) Inventors: David Maimon, Atlit (IL); Tamir S. Levi, Zikhron Yaakov (IL); Nikolai Gurovich, Hadera (IL); Karin Lavon, Tel-Aviv (IL); Martyn Rhys Thomas, San Juan Capistrano, CA (US)

(73) Assignee: EDWARDS LIFESCIENCES CORPORATION, Irvine, CA (US)

(21) Appl. No.: 18/961,277

(22) Filed: Nov. 26, 2024

Related U.S. Application Data
(63) Continuation of application No. PCT/US2023/025495, filed on Jun. 16, 2023.
(60) Provisional application No. 63/354,812, filed on Jun. 23, 2022.

(51) Int. Cl. A61F 2/24

(52) U.S. Cl. CPC A61F 2/2418; A61F 2250/0037; A61F 2250/0089

(57) ABSTRACT

The present disclosure relates to prosthetic valves that include information indicators which can indicate suitable information associated with a previously implanted prosthetic valve. Such information indicators can be internally formed in relatively wide support posts of mechanically expandable prosthetic valve, or can be externally formed along support posts of mechanically expandable prosthetic valve or vertical struts of balloon expandable prosthetic valves.

### CROSS-REFERENCE TO RELATED APPLICATIONS

This application is a continuation of International Application No. PCT/US2023/025495, filed Jun. 16, 2023, which claims the benefit of U.S. Provisional Application No. 63/354,812, filed Jun. 23, 2022, which is incorporated by reference herein.

### FIELD

The present disclosure relates to prosthetic valves that include information indicators which can indicate suitable information associated with a previously implanted prosthetic valve.

### BACKGROUND

Native heart valves, such as the aortic, pulmonary and mitral valves, function to assure adequate directional flow from and to the heart, and between the heart's chambers, to supply blood to the whole cardiovascular system. Various valvular diseases can render the valves ineffective and require replacement with artificial valves. Surgical procedures can be performed to repair or replace a heart valve. Surgeries are prone to an abundance of clinical complications, hence alternative less invasive techniques of delivering a prosthetic valve over a catheter and implanting it over the native malfunctioning valve, have been developed over the years.

Different types of prosthetic valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

Sometimes a need for re-intervention may arise after the patient has already had an earlier prosthetic valve implanted. In such cases, the clinician needs to know certain characteristics of the previously implanted prosthetic valve to determine the most appropriate course of action. Thus, a need exists for conveying such characteristics, that can relate, for example, to the manufacturer, type, model, valve-size, or any other characteristic of the originally implanted valve to guide the clinician in selecting a proper type of treatment, such as an appropriate replacement prosthetic valve.

### SUMMARY

The present disclosure is directed toward prosthetic valves that include information indicators which can indicate suitable information associated with a previously implanted prosthetic valve. Such information indicators can be internally formed in relatively wide support posts of mechanically expandable prosthetic valve, or can be externally formed along support posts of mechanically expandable prosthetic valve or vertical struts of balloon expandable prosthetic valves.

According to one aspect of the disclosure, a prosthetic valve comprises an annular frame movable between a compressed diameter in a radially compressed state and a deployed diameter in a radially expanded state. The frame comprises a plurality of intersecting angled struts, a plurality of vertical struts, at least one externally formed information indicator formed along at least one of the vertical struts, and a valvular structure mounted within the frame and comprising a plurality of leaflets configured to regulate flow through the prosthetic valve.

In some aspects, each of the plurality of vertical struts extends between a vertical strut inflow end and an opposite vertical strut outflow end.

In some aspects, the plurality of vertical struts comprises a plurality of commissure struts, each commissure strut comprising a commissure window and a plurality of non-commissural vertical struts, wherein each non-commissural vertical strut is devoid of a commissure window.

In some aspects, two of the angled struts intersect with each vertical strut inflow end, and another two of the angled struts intersect with each vertical strut outflow end.

In some aspects, the width of the externally formed information indicator is at least as great as the width of the vertical strut it is formed along.

In some aspects, the externally formed information indicator extends from the vertical strut inflow end of at least one of the commissure struts.

In some aspects, the externally formed information indicator extends from the vertical strut outflow end of at least one of the commissure struts.

In some aspects, the at least one externally formed information indicator comprises at least two externally formed information indicators, one of which extends from the vertical strut inflow end of at least one of the commissure struts, and the other extends from the vertical strut outflow end of the same commissure strut.

In some aspects, the width of the externally formed information indicator is greater than the width of the vertical strut it is formed along.

In some aspects, the at least one externally formed information indicator comprises a plurality externally formed information indicators, at least two of which are differently shaped from each other.

In some aspects, at least one of the externally formed information indicators is in the shape of a letter, and wherein one other of the externally formed information indicators is in the shape of a digit.

In some aspects, the frame further comprises at least two support struts extending between at least two adjacent vertical struts and intersecting with each other at a support apex, and wherein at least one support strut extends from the at least one externally formed information indicator.

In some aspects, the at least one externally formed information indicator comprises an inflow portion coinciding with the vertical strut inflow end, an opposite outflow portion, and a central portion disposed therebetween, wherein the central portion is in the form of a laterally oriented extension.

In some aspects, the at least one externally formed information indicator comprises an inflow portion connected to two of the angled struts, an opposite outflow portion, and a central portion disposed therebetween, wherein the central portion comprises a lateral body extending in a lateral direction and terminating at a bi-directional vertical extension, forming two shoulders at the transition between the lateral body and the bi-directional vertical extension.

In some aspects, the at least one externally formed information indicator extends from the vertical strut outflow end of one of the non-commissural vertical strut which is adjacent one of the commissure struts, such that an inflow portion of the externally formed information indicator is axially distal to an outflow edge of the commissure window.

In some aspects, an axial distance between the inflow portion of the externally formed information indicator and the outflow edge of the commissure window is less than the difference between an axial height of the prosthetic valve in the radially compressed state and an axial height of the prosthetic valve in the radially expanded state.

In some aspects, the at least one externally formed information indicator includes a first externally formed information indicator formed along one of the vertical struts, and a second externally formed information indicator formed along an adjacent vertical strut, wherein the first externally formed information indicator denotes a first digit of the deployed diameter of the prosthetic valve, and the second externally formed information indicator denotes a second digit of the deployed diameter of the prosthetic valve.

According to one aspect of the disclosure, a prosthetic valve comprises an annular frame movable between a compressed diameter in a radially compressed state and a deployed diameter in a radially expanded state. The frame comprises a plurality of actuation posts comprising upper post members and lower post members, a plurality of support posts, a plurality of actuators coupled to the actuation posts, the actuators configured to adjust the frame between the radially compressed state and the radially expanded state, at least one information indicator, and a valvular structure mounted within the frame and comprising a plurality of leaflets configured to regulate flow through the prosthetic valve.

In some aspects, each support post extends between a post inflow end and an opposite post outflow end.

In some aspects, the at least one information indicator is formed in or along at least one of the support posts.

In some aspects, the plurality of support posts comprises a plurality of commissure support posts, wherein each commissure support post comprises a commissure window and a plurality of non-commissural support posts, wherein each non-commissural support post is devoid of a commissure window.

In some aspects, the plurality of curved struts extend circumferentially between adjacent actuation posts and support posts and interconnect the actuation posts and the support posts.

In some aspects, two of the curved struts intersect with each post inflow end, and another two of the curved struts intersect with each post outflow end.

In some aspects, each support post intersects with at least eight curved struts extending from adjacent actuation posts.

In some aspects, the information indicator is an internally formed information indicator.

In some aspects, the width of the support post is greater than the width of the internally formed information indicator formed therein.

In some aspects, the at least one internally formed information indicator comprises at least two internally formed information indicators, both of which are formed at different axial positions of at least one of the non-commissural support posts.

In some aspects, the information indicator is an externally formed information indicator.

In some aspects, the width of the externally formed information indicator is greater than the width of the support post it is formed along.

In some aspects, the at least one information indicator includes a first information indicator in one of the support posts, and a second information indicator in an adjacent support post, wherein the first information indicator denotes a first digit of the deployed diameter of the prosthetic valve, and the second information indicator denotes a second digit of the deployed diameter of the prosthetic valve.

According to some aspects of the disclosure, there is provided a prosthetic valve comprising an annular frame movable between a compressed diameter in a radially compressed state and a deployed diameter in a radially expanded state. The frame comprises a plurality of intersecting angled struts and a plurality of vertical struts, each extending between a vertical strut inflow end and an opposite vertical strut outflow end. Two of the angled struts intersect with each vertical strut inflow end, and another two of the angled struts intersect with each vertical strut outflow end. The frame further comprises at least one information indicator formed in or along at least one of the vertical struts.

According to some aspects of the disclosure, there is provided a prosthetic valve comprising an annular frame and a valvular structure mounted within the frame. The frame is movable between a compressed diameter in a radially compressed state and a deployed diameter in a radially expanded state. The frame comprises a plurality of intersecting angled struts and a plurality of vertical struts, wherein each vertical strut extends between a vertical strut inflow end and an opposite vertical strut outflow end. The plurality of vertical struts comprises a plurality of commissure struts and a plurality of non-commissural vertical struts.

Each commissure strut comprises a commissure window. Each non-commissural vertical strut is devoid of a commissure window. The frame further comprises at least one externally formed information indicator formed along at least one of the vertical struts. The valvular structure comprises a plurality of leaflets configured to regulate flow through the prosthetic valve. Two of the angled struts intersect with each vertical strut inflow end, and another two of the angled struts intersect with each vertical strut outflow end. The width of the externally formed information indicator is at least as great as the width of the vertical strut it is formed along

According to some aspects of the disclosure, there is provided a prosthetic valve comprising an annular frame and a valvular structure mounted within the frame. The frame is movable between a compressed diameter in a radially compressed state and a deployed diameter in a radially expanded state. The frame comprises a plurality of actuation posts, a plurality of support posts, a plurality of curved struts, a plurality of actuators coupled to the actuation posts, and at least one information indicator formed in or along at least one of the support posts. Each support post extends between a post inflow end and an opposite post outflow end. The plurality of support posts comprises a plurality of commissure support posts and a plurality of non-commissural support posts. Each commissure support post comprises a commissure window. Each non-commissural support post is devoid of a commissure window.

The curved struts extend circumferentially between adjacent actuation posts and support posts, and interconnect the actuation posts and the support posts. The actuators are configured to adjust the frame between the radially compressed state and the radially expanded state. Two of the curved struts intersect with each post inflow end, and another two of the curved struts intersect with each vertical strut outflow end. Each support post intersects with at least eight curved struts extending from adjacent actuation posts.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1 is perspective view of an exemplary mechanically expendable prosthetic valve.

FIG. 2 is perspective view of a frame of another exemplary mechanically expendable prosthetic valve.

FIG. 3 shows an exemplary delivery apparatus carrying a mechanically expandable prosthetic valve.

FIG. 4 is perspective view of a frame of another exemplary mechanically expendable prosthetic valve.

FIG. 5A is perspective view of an exemplary balloon expendable prosthetic valve.

FIG. 5B is a perspective view of the frame of the prosthetic valve of FIG. 5A.

FIG. 6 shows an exemplary delivery apparatus carrying a balloon expandable prosthetic valve.

FIG. 7A is perspective view of a frame of another exemplary balloon expendable prosthetic valve.

FIG. 7B shows a portion of the frame of FIG. 7A in a flattened configuration.

FIG. 8A is perspective view of a frame of another exemplary balloon expendable prosthetic valve.

FIG. 8B shows a portion of the frame of FIG. 8A in a flattened configuration.

FIG. 9 shows a portion of a frame of another exemplary balloon expendable prosthetic valve.

FIG. 10 is perspective view of the prosthetic valve of FIG. 9.

FIG. 11 shows a portion of a frame of another exemplary balloon expendable prosthetic valve.

FIG. 12A is perspective view of a frame of another exemplary balloon expendable prosthetic valve.

FIG. 12B shows a portion of the frame of FIG. 12A in a flattened configuration, with zoomed-in regions of two information indicators thereof.

FIG. 13 shows a portion of a frame of another exemplary balloon expendable prosthetic valve.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/of” means “and” or “of”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

It should be understood that the disclosed examples can be adapted to deliver and implant prosthetic devices in any of the native annuluses of the heart (e.g., the aortic, pulmonary, mitral, and tricuspid annuluses), and can be used with any of various delivery approaches (e.g., retrograde, antegrade, transseptal, transventricular, transatrial, etc.).

In order to avoid undue clutter from having too many reference numbers and lead lines on a particular drawing, some components will be introduced via one or more drawings and not explicitly identified in every subsequent drawing that contains that component.

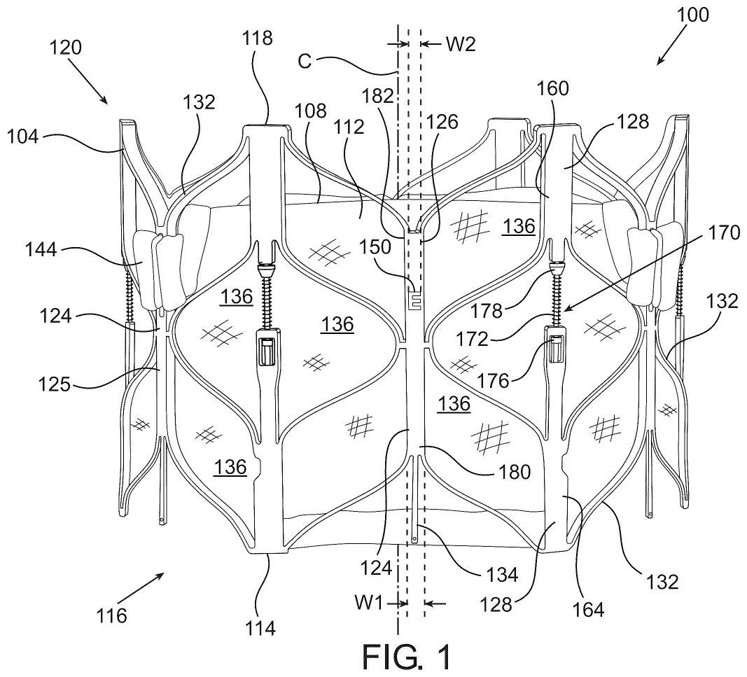

FIG. 1 illustrates a prosthetic valve 100, according to one example. The prosthetic valve 100 can be configured to replace a native heart valve (e.g., aortic, mitral, pulmonary, and/or tricuspid valves). The prosthetic valve 100 is illustrated as a mechanically expandable prosthetic valve that can be radially compressed for delivery to an implantation location within a patient's body and then radially expanded to a working diameter at the implantation location. The prosthetic valve 100 can include a frame 104 having an annular shape. The prosthetic valve 100 can further include a valvular structure 108 supported within and coupled to the frame 104.

In the example, the valvular structure 108 includes one or more leaflets 112 made of flexible material and configured to open and close to regulate blood flow. In one example, the valvular structure 108 can have three leaflets 112, which can be arranged to collapse in a tricuspid arrangement. The leaflets 112 can be made in whole or in part from pericardial tissue (e.g., bovine pericardial tissue), biocompatible synthetic materials, or various other suitable natural or synthetic materials.

As illustrated, the frame 104 has an inflow end 116, an outflow end 120, and a central longitudinal axis C extending in a direction from the inflow end 116 to the outflow end 120. The frame 104 can include a plurality of support posts 124 and actuation posts 128 aligned with the central longitudinal axis C and spaced along a circumference of the frame 104. In one example, the support posts 124 and actuation posts 128 can be arranged in an alternating manner along the circumference of the frame 104. The frame 104 can further include a plurality of struts 132 extending circumferentially between adjacent support posts 124 and actuation posts 128 and interconnecting the support posts 124 and actuation posts 128. The struts 132, support posts 124, and actuation posts 128, define cells 136 of the frame 104. As illustrated, the struts 132 can have an angled or curved shape.

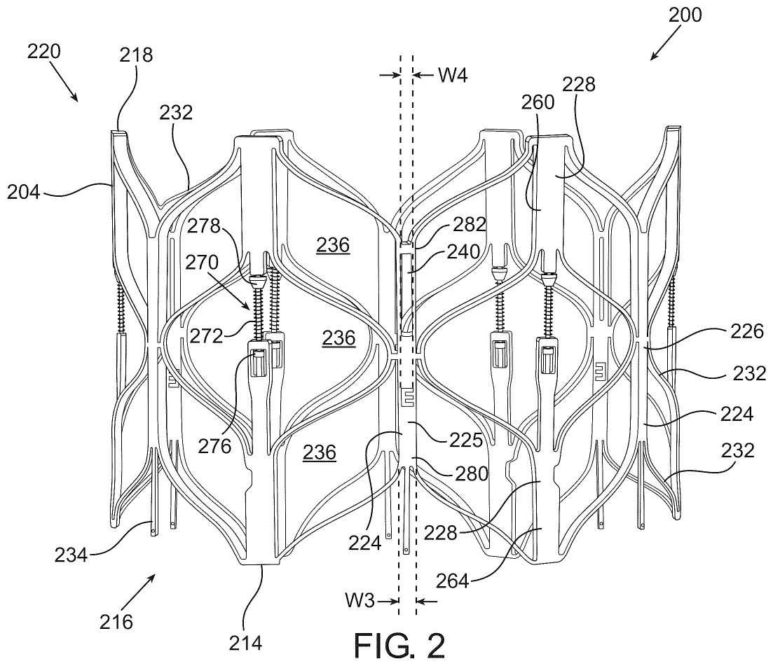

One or more commissure windows 140 can be formed in one or more of the support posts 124 (commissure window 140 is concealed in FIG. 1, but is similar to commissure window 240 exposed in FIG. 2). Commissures 144 can be formed at the commissure windows 140 to couple the leaflets 112 to the frame 104. Support posts 124 can include commissure support posts 125, which are support posts 124 that include commissure windows 140, and non-commissural support posts 126, which are support posts 124 devoid of commissure windows. The commissure support posts 125 and non-commissural support posts 126 can be arranged in an alternating manner along the circumference of the frame 104. In the illustrated example, frame 104 includes a total of six support posts 124, three of which are commissure support posts 125 and three of which are non-commissural support posts 126.

One or more of the support posts 124 can further include cantilevered struts 134 extending to the inflow end 116 of the frame 104. In some examples, the cantilevered struts 134 can extend such that distal ends of the cantilevered struts 134 align with or substantially align with the inflow end 116 of the frame 104.

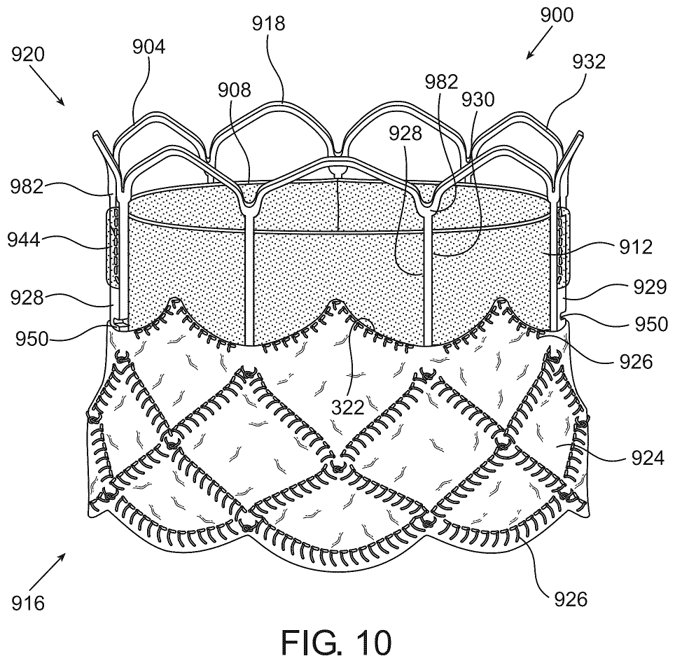

The prosthetic valve 100 can further include one or more skirts or sealing members. For example, the prosthetic valve 100 can include an inner skirt (not shown in FIG. 1), mounted on the radially inner surface of the frame 104. The inner skirt can function as a sealing member to prevent or decrease perivalvular leakage, to anchor the leaflets 112 to the frame 104, and/or to protect the leaflets 112 against damage caused by contact with the frame 104 during crimping and during working cycles of the prosthetic valve 100. The prosthetic valve 100 can further include an outer skirt (not shown in FIG. 1, but can be generally similar to outer skirt 924 shown in FIG. 10 for a different valve type) mounted on the outer surface of the frame 104. The outer skirt can function as a sealing member for the prosthetic valve 100 by sealing against the tissue of the native valve annulus and helping to reduce paravalvular leakage past the prosthetic valve 100. The inner and outer skirts can be formed from any of various suitable biocompatible materials, including any of various synthetic materials, including fabrics (e.g., polyethylene terephthalate fabric) or natural tissue (e.g., pericardial tissue). Further details regarding the use of skirts or sealing members in prosthetic valves can be found, for example, in U.S. Patent Application No. 62/854,702 and PCT Patent Application No. US2020/024559, each of which is incorporated by reference herein.

In some cases, as shown in the example illustrated in FIG. 1, inflow edge portions of the leaflets 112 can be attached to the cantilevered struts 134 and/or to selected struts 132 of the frame 104. Alternatively or additionally, cantilevered struts 134 can prevent or mitigate portions of an outer skirt from extending radially inwardly and thereby prevent or mitigate any obstruction of flow through the inflow end 116 of the frame 104 caused by the outer skirt. The cantilevered struts 134 can further serve as supports to which portions of the inner and/or outer skirts can be coupled. For example, sutures used to connect the inner and/or outer skirts can be wrapped around the cantilevered struts 134 and/or can extend through apertures formed at end portions of the cantilevered struts 134.

In some implementations, leaflets 112 can be sutured directly to struts 132 of the frame 104. In other implementation, proximal edge portions of the leaflets can be sutured to an inner skirt generally along the scallop line. The inner skirt can in turn be sutured, via one or more sutures, for example, to adjacent struts 132 of the frame 104.

Each support post 124, including any commissure support posts 125 or non-commissural support post 126, extends between a post inflow end 180 which is closer to the inflow end 116 of the valve 100, and a post outflow end 182 which is closer to the outflow end 120 of the valve 100. Two curved struts 132 intersect with each support post 124 at a post inflow end 180, which is circumferentially disposed between two adjacent inflow apices 114, such that a corresponding cantilevered strut 134 can extend distally from the post inflow end 180. Similarly, two curved struts 132 intersect with each support post 124 at a post outflow end 182, which is circumferentially disposed between two adjacent outflow apices 118.

As further shown, each commissure support post 125 and each non-commissural support post 126 also intersects, at a middle portion thereof, with two four additional curved struts 132 extending from adjacent upper post members 160 and lower post members 164 on both sides, resulting in each support posts 124, and specifically, each commissure support post 125 and each non-commissural support post 126, intersecting with a total of at least eight curved struts extending from adjacent actuation posts 128.

In one example, the frame 104 can be adjusted between a radially expanded configuration and a radially compressed configuration by deflecting the struts 132. In one example, the frame 104 (e.g., the posts and struts) can be made of biocompatible plastically-expandable materials that will allow the frame 104 to be adjusted between the radially expanded configuration and radially compressed configuration. Suitable examples of plastically-expandable materials that can be used in forming the frame 104 include, but are not limited to, stainless steel, cobalt chromium alloy, and/or nickel titanium alloy (which can also be referred to as “NiTi” or “nitinol”).

In some examples, one or more actuators 170 can be coupled to the actuation posts 128, and used to adjust the frame 104 between the radially expanded configuration and the radially compressed configuration. In one example, each actuation post 128 can include an upper post member 160 and a lower post member 164 (the terms “upper” and “lower” are relative to the orientation of the prosthetic valve 100 in FIG. 1) aligned with the longitudinal axis C and having opposing ends separated by a gap. The respective actuator 170 can be coupled to the post members 160, 164 and operable to increase or decrease the gap therebetween in order to radially compress or expand the frame 104. Struts 132 can converge with upper post members 160 to define outflow apices 118 at the outflow end 120. Struts 132 can similarly converge with lower post members 164 to define inflow apices 114 at the inflow end 116.

In one example, the actuator 170 can include an actuator rod 172 with an attached actuator head. In the example illustrated in FIG. 1, the actuator rod 172 extends through or into the post members 160, 164 and across the gap therebetween. In the example illustrated in FIG. 1, the actuator rod 172 is inserted into the upper post member 160 from the outflow end 120, and the actuator head (hidden from view in FIG. 1) can be disposed or retained at the outflow apex of the upper post member 160.

In some examples, the actuator rod 172 is externally threaded. As illustrated in FIG. 1, the lower post member 164 can include a nut 176 with an internal thread to threadedly engage the actuator rod 172. In this case, the actuator rod 172 can be axially translated by rotating the actuator rod 172 relative to the nut 176. In some examples, the actuator rod 172 can be freely slidable relative to the upper post member 160. In other examples, the actuator rod 172 can threadedly engage the upper post member 160. The term “axially translated”, as used herein, refers to translation along an axis coinciding with or parallel to the central longitudinal axis C.

In one scenario, the actuator rod 172 can be rotated in a first direction to move the upper post member 160 towards the lower post member 164 and thereby decrease the size of the gap therebetween, which can have the effect of radially expanding the frame 104. In another scenario, the lower post member 164 may be held steady while the actuator rod 172 is rotated in a second direction to move the upper post member 160 away from the lower post member 164 and thereby increase the size of the gap therebetween, which can have the effect of radially compressing the frame 104.

The actuator rod 172 also can include a stopper 178 (e.g., in the form of a nut, washer or flange) disposed thereon. The stopper 178 can be disposed on actuator rod 172 such that it sits within the gap therebetween. Further, the stopper 178 can be integrally formed on or fixedly coupled to the actuator rod 172 such that it does not move relative to the actuator rod 172. Thus, the stopper 178 can remain in a fixed axial position on the actuator rod 172 such that it moves in lockstep with the actuator rod 172.

When the actuator rod 172 is rotated in a direction configured to collapse the prosthetic valve, the stopper 178 moves toward the outflow end 120 of the frame until the stopper 178 abuts the inflow end of the upper post member 160. Upon further rotation of the actuator rod 172, the stopper 178 can apply a proximally directed force to the upper post member 160 to radially compress the frame 104. Specifically, during crimping/radial compression of the prosthetic valve 100, the actuator rod 172 can be rotated in a direction that causes the stopper 178 to push against (i.e., provide a proximally directed force to) the inflow end of the upper post member 160, thereby causing the upper post member 160 to move away from the lower post member 164, and thereby axially elongating and radially compressing the prosthetic valve 100.

In an alternative implementation, some of the actuator rods 172 can be rotated in one direction while the other actuator rods 172 are rotated in an opposite direction simultaneously to either radially expand the frame or radially compress the frame. This counter-rotation of the actuator rods can be used to help reduce the likelihood of the entire frame 104 rotating about the central longitudinal axis C during rotation of the actuator rods 172 about their respective axes (e.g., when radially expanding the frame 104).

Additional examples of mechanically expandable valves can be found in International Application No. PCT/US2021/052745 and U.S. Provisional Applications Nos. 63/209,904 and 63/282,463, which are incorporated by reference herein.

Prosthetic valves disclosed herein, include at least one information indicator formed in or along a portion of a support post or vertical strut of the frame. Since the frame of a prosthetic valve is conventionally made of a material which is radiopaque (e.g., metallic materials), the boundaries of any such information indicator, either formed from opening passing through the thickness of components of the frame (such as support posts) or formed by cutting the outer boundaries of components of the frame (such as vertical struts) to form a desired shape of the indicator, can be detected using suitable clinical imaging techniques, such as fluoroscopy, magnetic resonance imaging (MRI), computed tomography (CT), etc.

The information indicator can provide a clinician (or any other user) information regarding the implanted prosthetic valve, e.g., one or more of a manufacturer, type, model number, valve-size and/or manufacture date associated with the prosthetic valve. If re-intervention is required, such information can aid a clinician in determining which replacement prosthetic valve can be compatible with the original prosthetic valve, for example. In another scenario, such information can be of importance in emergency situations to aid in determining whether a patient has an implanted prosthetic valve, and if so, the manufacturer, type, model, etc. of the prosthetic valve. Any information indicator disclosed herein can be in the form of an alphanumeric character, a symbol, or any other geometric shape.

For example, a manufacturer may have multiple models of prosthetic valves, each of which may come in a number of sizes (e.g., 20 mm, 23 mm, 26 mm, 29 mm), which can be expanded to a deployed diameter (e.g., 23 mm for a 23 mm valve) or a range of diameters (e.g., 21-24 mm for a 23 mm valve). An information indicator can be indicative of the implanted valve diameter, for example by being in the shape of the last digit of the valve size (e.g., “3” for a 23 mm valve). In other examples, two adjacent information indicators can be formed as both digits of the valve size (for example, information indicators shaped as “2” and “3” for a 23 mm valve).

As shown, the actuation posts 128 are arranged in pairs, each pair including an upper post member 160 and a lower post member 164 which can be axially aligned with each other, and each pair of actuation posts 128 can be connected, such as via struts 132, to a commissure support post 125 on one side thereof, and to a non-commissural support post 126 on the other side. As shown, support posts 124 disposed between struts 132 can have a width, in the circumferential direction, that is greater than the width of other curved or angled struts 132 of the frame 104, so as to improve structural stability to the frame 104. This unique feature of increased width can be taken advantage of, by adding an information indicator 150 formed of openings passing through the thickness of one or more support post 124, bound between the outer borders of the support post 124.

FIG. 1 shows a mechanically expandable prosthetic valve 100 with at least one non-commissural support post 126 that includes an internally formed information indicator 150.

The term “internally formed”, as used herein throughout the specification, refers to being formed of openings or cutouts passing through a thickness of the support post or vertical strut (as described further below with respect to balloon expandable prosthetic valves), such that the outer boundaries of the openings forming the information indicator are completely bound within the outer boundaries of the support post or vertical strut, and more specifically, such that the outer boundaries of the information indicator to not form circumferential boundaries of any portion of the support post or vertical strut, but are rather offset away from the circumferential boundaries of the support post or vertical strut, leaving frame material bound between the circumferential boundaries of the information indicator and the circumferential boundaries of the support post or vertical strut in which it is formed. Otherwise stated, an internally formed information indicator is formed by creating one or more through-openings or voids in the shape of the desired alphanumeric character, symbol, or any other geometric shape of the information indicator.

As shown in FIG. 1, the non-commissural support post 126 can have a width W1 in the circumferential direction (i.e., between its circumferential boundaries), while the information indicator 150 formed therein can have a width W2 in the circumferential direction (i.e., between the indicator's circumferential boundaries), such that W1 is greater than W2 (W1>W2). As mentioned, the specific frame design shown in FIG. 1 and described herein, has the advantage of including support posts 124, such as non-commissural support posts 126, having a width which is significantly greater than that of other curved struts 132 of the frame 104 (for example, W1 can be at least three times greater than the width of curved or angled struts 132), allowing internally-formed information indicators to be formed in such support posts 124 without needing to change the external shape or dimensions of the support posts 124 or any other components of the prosthetic valve 100 for that end.

The information indicator 150 can be formed at any location along the length of the non-commissural support post 126. While an E-shaped information indicator 150 is illustrated, it is to be understood that the information indicator 150 can be in the shape of any other alphanumeric character, symbol, or any other geometric shape. While a single information indicator 150 if shown to be formed in one non-commissural support post 126 in FIG. 1, it is to be understood that more than one non-commissural support post 126 can include an internally formed information indicator 150, and that any non-commissural support post 126 can include more than one internally formed information indicator 150. When a plurality of internally formed information indicators 150 are included, they can be similarly shaped, or differently shaped—for example to convey different information details of the prosthetic valve 100.

As mentioned above, the size, i.e., deployed diameter, of a prosthetic valve, can include two digits. In some implementations, at least one internally formed information indicators denotes the deployed diameter of the prosthetic valve by taking the form of the last digit of the deployed diameter of the prosthetic valve. Since a support post, such as an internally formed non-commissural support post 126, may not provide sufficient space to include both digits, in some implementations, two adjacent support posts, such as two adjacent non-commissural support post 126, can each include one of the digits of a two-digit deployed diameter, which can be similar or different from each other. For example, for a prosthetic valve having a deployed diameter of 23 mm, one internally formed information indicator formed in a first non-commissural support post 126 can take the form of the first digit (e.g., “2”), while another internally formed information indicator formed in a second non-commissural support post 126, adjacent the first non-commissural support post 126, can take the form of the second digit (e.g., “3”).

FIG. 2 illustrates another exemplary prosthetic valve 200, shown without soft components (such as skirts or the valvular structure) for clarity. Prosthetic valve 200 can be structurally similar to and function in a similar manner as the described prosthetic valve 100, except that prosthetic valve 200 is shown to have at least one commissure support post 225 that includes an internally formed information indicator 250. For instance, the prosthetic valve 200 can include a frame 204 extending between an inflow end 216 and an outflow end 220, the frame 204 including a plurality of actuation support posts 228 defining pairs of upper post members 260 and lower post members 264, and support posts 224 that include commissure support posts 225 equipped with commissure windows 240, and non-commissural support posts 226 devoid of commissure windows. The frame 204 can also include a plurality of curved or angled struts 232 extending circumferentially between adjacent support posts 224, 228 and interconnecting the support posts 224, 228, wherein the struts 232 and the support posts 224, 228 define cells 236 of the frame 204.

Each support post 224, including any commissure support posts 225 or non-commissural support post 226, extends between a post inflow end 280 and a post outflow end 282. Two curved struts 232 intersect with each support post 224 at a post inflow end 280, which is circumferentially disposed between two adjacent inflow apices 214, such that a corresponding cantilevered strut 134 can extend distally from the post inflow end 180. Similarly, two curved struts 232 intersect with each support post 224 at a post outflow end 282, which is circumferentially disposed between two adjacent outflow apices 218. Other structural and functional components of the prosthetic valve 200 can be similar to those described above with respect to prosthetic valve 100, with like numbers referring to like components, and in the interest of brevity will not be further described.

As shown in FIG. 2, the commissure support post 225 can have a width W3 in the circumferential direction, while the information indicator 250 formed therein can have a width W4 in the circumferential direction, such that W3 is greater than W4 (W3>W4). Non-commissural support posts 226 can have a width W1 similar to non-commissural support posts 126 described above with respect to frame 104. The width W4 of commissure support post 225 can be similar to the width W1 of the non-commissural support post 226 for the same reasons described above (i.e., increasing structural stability of the frame 204), or it can be, in some implementations, greater that the width W1 of the non-commissural support post 226, for example due to the fact that it needs to accommodate a commissure window 240 formed therein, which is absent from non-commissural support post 226.

While information indicator 250 is shown to be distal to the commissure window 240 in the illustrated example, it is to be understood that an internally formed information indicator 250 can be formed at any location along the length of the commissure support post 225, including proximal to a commissure window 240 if the commissure window 240 is sufficiently distanced from the post outflow end 282 to accommodate such an information indicator 250.

While an E-shaped information indicator 250 is illustrated, it is to be understood that the information indicator 250 can be in the shape of any other alphanumeric character, symbol, or any other geometric shape. While a single information indicator 250 if shown to be formed in each of the three commissure support post 225 illustrated in FIG. 2, it is to be understood that any other number of commissure support posts 225 can include an internally formed information indicator 250, and that any commissure support post 225 can include more than one internally formed information indicator 250. When a plurality of internally formed information indicators 250 are included, they can be similarly shaped, or differently shaped—for example to convey different information details of the prosthetic valve 200.

While prosthetic valve 100 is shown to include at least one internally formed information indicator 150 in at least one non-commissural support post 126 in FIG. 1, and prosthetic valve 200 is shown to include internally formed information indicators 250 in commissure support posts 225 in FIG. 2, it is to be understood that in other implementations, a mechanically expandable prosthetic valve 100 or 200 can include at least one internally formed information indicator 150, 250 in at least one non-commissural support post 126, 226 thereof, together with at least one internally formed information indicator 150, 250 in at least one commissure support post 125, 225 thereof, respectively. In such implementations, some or all of the plurality of internally formed information indicator 150, 250 can be similarly shaped or differently shaped.

As mentioned above, since a support post 124, 224, such as a commissure support post 125, 225 or a non-commissural support post 126, 126, may not provide sufficient space to include both digits, in some implementations, two adjacent support posts 124, 224, such as a commissure support post 125, 225 and an adjacent non-commissural support post 126, 226, can each include one of the digits of a two-digit deployed diameter, which can be similar or different from each other. For example, for a prosthetic valve having a deployed diameter of 23 mm, one internally formed information indicator formed in a commissure support post 125, 225 can take the form of the first digit (e.g., “2”), while another internally formed information indicator formed in a non-commissural support post 126, 226, adjacent the commissure support post 125, 225, can take the form of the second digit (e.g., “3”).

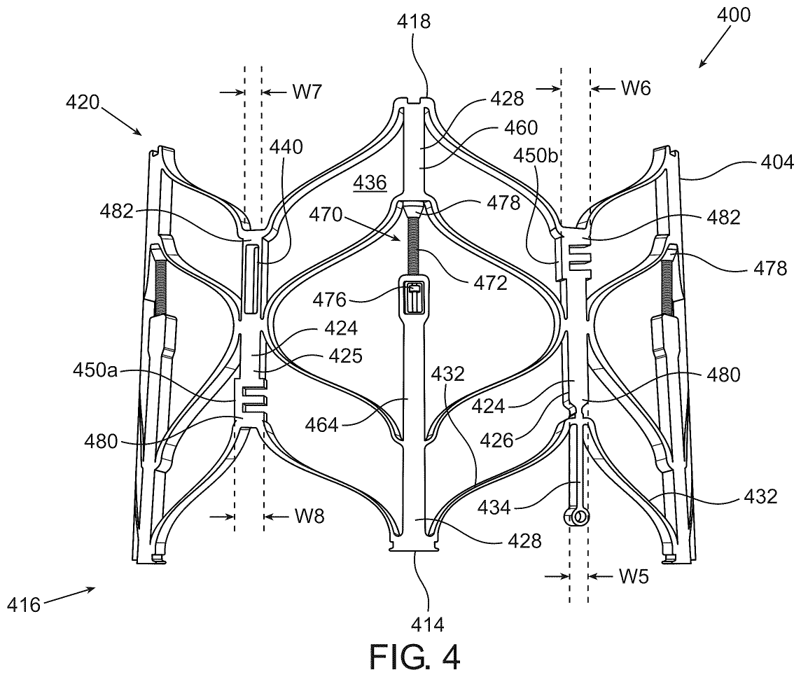

FIG. 4 illustrates another exemplary prosthetic valve 400, shown without soft components (such as skirts or the valvular structure) for clarity. Prosthetic valve 400 can be structurally similar to and function in a similar manner as the described prosthetic valve 100, except that prosthetic valve 400 can include one or more support posts 424 provided with at least one externally formed information indicator 450 instead of internally formed information indicators 150, 250. For instance, the prosthetic valve 400 can include a frame 404 extending between an inflow end 416 and an outflow end 420, the frame 404 including a plurality of actuation support posts 428 defining pairs of upper post members 460 and lower post members 464, and support posts 424 that include commissure support posts 425 equipped with commissure windows 440, and non-commissural support posts 426 devoid of commissure windows. The frame 404 can also include a plurality of curved or angled struts 432 extending circumferentially between adjacent support posts 424, 428 and interconnecting the support posts 424, 428, wherein the struts 432 and the support posts 424, 428 define cells 436 of the frame 404.

While only one side of the frame 404 is depicted in FIG. 4, it should be appreciated that the frame 404 forms an annular structure having an opposite side that is substantially identical to the portion shown in FIG. 4. Each support post 424, including any commissure support posts 425 or non-commissural support post 426, extends between a post inflow end 480 and a post outflow end 482. Two curved struts 432 intersect with each support post 424 at a post inflow end 480, which is circumferentially disposed between two adjacent inflow apices 414, such that a corresponding cantilevered strut 434 can extend distally from the post inflow end 480.

Similarly, two curved struts 432 intersect with each support post 424 at a post outflow end 482, which is circumferentially disposed between two adjacent outflow apices 418. Other structural and functional components of the prosthetic valve 400 can be similar to those described above with respect to prosthetic valve 100, with like numbers referring to like components, and in the interest of brevity will not be further described.

As mentioned, a mechanically expandable prosthetic valve 400 has at least one support post 424 that includes an externally formed information indicator 450. The term “externally formed”, as used herein throughout the specification, refers to being formed by a cutout along the outer borders that define at least a portion of the support post or vertical strut, such that the outer boundaries forming the information indicator are continuous with the outer boundaries defining the remainder of the support post or vertical strut. Otherwise stated, an externally formed information indicator is formed by creating a cutout along the outer boundaries such that the solid material defined between these boundaries is in the shape of the desired alphanumeric character, symbol, or any other geometric shape of the information indicator. An externally formed information indicator of the current specification will have a width, in the circumferential direction, which is equal to or greater than the width of the remainder of the support post or vertical strut.

An externally formed information indicator can be advantageous over an internally formed information indicator when support posts or support struts are not wide enough (in the circumferential direction) to accommodate internally formed information indicator, or when the resulting potential internally formed information indicator is relatively small due to the restricted width of the support post or vertical strut in which it is to be formed, making it harder to identify under clinical imaging techniques (e.g., fluoroscopy), since an externally formed information indicator can have a width which is greater than that of an optional alternative internally formed information indicator, such as having a width similar to that of the support post or vertical strut, or a width which is greater than that of the support post or vertical strut.

An externally formed information indicator of the current specification will extend from a post inflow end or a post outflow end of a support post, or from a vertical strut inflow end or a vertical strut outflow end of a vertical strut (described in further detail below). The position of an externally formed information indicator, extending from an inflow or outflow end of a support post or a vertical strut, is advantageous over other axial positions along the support post or vertical strut, since such inflow or outflow ends, along which curved or angled struts of the framer converge with the support posts or vertical struts, can provide increased structural stability to the resulting information indicator.

The frame 404 in FIG. 4 is illustrated to show two exemplary externally formed information indicators 450: an information indicator 450a extending from a post inflow end 480 of a commissure support post 424, and an information indicator 450b extending from a post outflow end 482 of a non-commissural support post 426. As shown, the non-commissural support post 426 can have a width W5 in the circumferential direction, while the information indicator 450b formed therealong can have a width W6 in the circumferential direction, such that W6 is greater than W5 (W6>W5). The commissure support post 425 can have a width W7 in the circumferential direction, while the information indicator 450a formed therealong can have a width W8 in the circumferential direction, such that W8 is greater than W7 (W8>W7). The width W7 of commissure support post 425 can be similar to the width W5 of the non-commissural support post 426, or it can be, in some implementations, greater that the width W5 of the non-commissural support post 426, for example due to the fact that it needs to accommodate a commissure window 440 formed therein, which is absent from non-commissural support post 426.

While information indicator 450a is shown to extend from a post inflow end 480 of a commissure support post 425, distal to the commissure window 440 in the illustrated example, it is to be understood that an externally formed information indicator 450 can similarly extend from a post outflow end 482 of a commissure support post 425, proximal to the commissure window 440, if the commissure window 440 is sufficiently distanced from the post outflow end 482 to accommodate such an information indicator 450. Similarly, while a commissure support post 425 is shown in the illustrated example to include a single externally formed information indicator 450a, in alternative implementations, a commissure support post 425 can include two externally formed information indicator 450 at opposite ends thereof, one extending from its post inflow end 480, and the other extending from its post outflow end 482.

While information indicator 450b is shown to extend from a post outflow end 482 of a non-commissural support post 426 in the illustrated example, it is to be understood that an externally formed information indicator 450 can similarly extend from a post inflow end 480 of a non-commissural support post 426. Similarly, while a non-commissural support post 426 is shown in the illustrated example to include a single externally formed information indicator 450b, in alternative implementations, a non-commissural support post 426 can include two externally formed information indicator 450 at opposite ends thereof, one extending from its post inflow end 480, and the other extending from its post outflow end 482.

While the frame 404 illustrated in FIG. 4 is shown to include both an information indicator 450a formed along a commissure support post 425 and an information indicator 450b formed along a non-commissural support post 426, it is to be understood that such a configuration is shown by way of illustration and not limitation, and that a frame 404 can be equipped with one or more commissure support posts 425 that include one or two externally formed information indicators 450, with all non-commissural post 426 being devoid of any information indicators, or a frame 404 can be equipped with one or more non-commissural post 426 that include one or two externally formed information indicators 450, with all commissure support post 425 being devoid of any information indicators.

While E-shaped information indicators 450a, 450b are illustrated, it is to be understood that any information indicator 450 can be in the shape of any other alphanumeric character, symbol, or any other geometric shape. When a plurality of information indicators are provided in a single frame 404, such as in the illustrated example, they can be similarly shaped, or differently shaped—for example to convey different information details of the prosthetic valve 400.

While prosthetic valve 100 and 200 are shown to include internally formed information indicators 150 and 250, and prosthetic valve 400 is shown to include externally formed information indicators 450, it is to be understood that in some implementations, a mechanically expandable prosthetic valve, similar to any of the prosthetic valve 100, 200, 400 disclosed hereinabove, can include a combination of at least one internally formed information indicator 150, 250 and at least one externally formed information indicator 450, formed within or along the same or different support struts. Such combined implementations can be useful if the internally formed information indicator is differently shaped than the externally formed information indicator, each indicator designed to convey different information detail, wherein an internally formed information indicator is in the form of an alphanumeric character, symbol, or any other geometric shape, which can be adequately formed within the boundaries of a support post without compromising the ability to properly identify it under clinical imaging techniques, while the externally formed information indicator is in the form of an alphanumeric character, symbol, or any other geometric shape which needs to be relatively wider to allow proper identification of its shape under clinical imaging techniques.

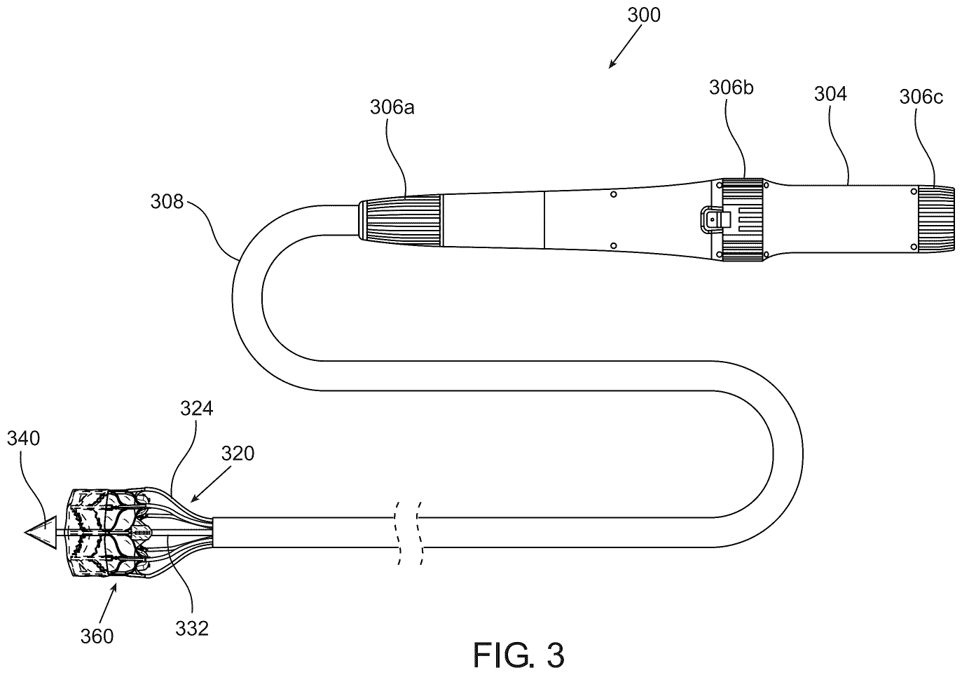

FIG. 3 illustrates a delivery apparatus 300, according to one configuration, adapted to deliver a mechanically expandable prosthetic valve 360 described herein (e.g., prosthetic valve 100, 200 and 400). The prosthetic valve 360 can be releasably coupled to the delivery apparatus 300. It should be understood that the delivery apparatus 300 can be used to implant prosthetic devices other than prosthetic valves, such as stents or grafts.

The delivery apparatus 300 in the illustrated example generally includes a handle 304, an outer elongated shaft 308 extending distally from the handle 304 and at least one actuator assembly 320 extending distally through the outer shaft 308. The delivery apparatus 300 can also include an elongated nosecone shaft 332 extending distally from the handle 304 through the outer shaft 308. A nosecone 340 can be connected to the distal end of the nosecone shaft 332. The at least one actuator assembly 320 can be configured to radially expand and/or radially collapse the prosthetic valve 360 when actuated.

As illustrated, one actuator assembly 320 can be provided for each actuator (e.g., actuator 170, 270 or 470) on the prosthetic valve 360. For example, six actuator assemblies 320 can be provided for a prosthetic valve 360 having six actuators. In other configurations, however, any greater or fewer number of actuator assemblies can be present.

The distal end portion of the shaft 308 can be sized and shaped to house the prosthetic valve 360 in a radially compressed, delivery state during delivery of the prosthetic valve through, for example, the vasculature of a patient. In this way, the distal end portion of shaft 308 functions as a delivery sheath or capsule for the prosthetic valve during delivery.

The actuator assemblies 320 can be releasably coupled to the prosthetic valve 360. For example, in the illustrated configuration, each actuator assembly 320 can be coupled to a respective actuator of the prosthetic valve 360. Each actuator assembly 320 can comprise a support tube 324, an actuator member (hidden within support tube 324 in FIG. 3), and optionally a locking tool. When actuated, the actuator assembly can transmit pushing and/or pulling forces to portions of the prosthetic valve to radially expand and collapse the prosthetic valve as previously described. The actuator assemblies 320 can be at least partially disposed radially within, and extend axially through, one or more lumens of the outer shaft 308. For instance, the actuator assemblies 320 can extend through a central lumen of the shaft 308 or through separate respective lumens formed in the shaft 308.

The terms “releasably coupled” or “releasably attached”, as used herein, are interchangeable, and refer to two components coupled in such a way that they are coupled together and can be separated without plastically deforming either of the components.

Although not illustrated, the delivery apparatus 300 can include, in some implementations, a multi-lumen delivery shaft extending through the lumen of the outer shaft and having a plurality of lumens therein. Any of the nosecone shaft 332 and/or actuation assemblies 320 can extend through lumens of the multi-lumen delivery shaft.

The actuator member of each actuator assembly 320 can be releasably coupled to a respective actuator of the prosthetic valve (e.g., actuator 170, 270 or 470). The support tube 324 of each actuator assembly 320 can abut an adjacent portion of the frame of the prosthetic valve, such as an outflow apex (e.g., apex 118, 218 or 418). In this manner, during valve expansion, the support tubes 324 can prevent movement of the outflow end of the prosthetic valve relative to the delivery apparatus while the actuator members of the actuator assemblies 320 can actuate the actuators of the prosthetic valve and cause the inflow end of the prosthetic valve to move toward the outflow end of the prosthetic valve.

The handle 304 of the delivery apparatus 300 can include one or more control mechanisms (e.g., knobs 306 or other actuating mechanisms) for controlling different components of the delivery apparatus 300 in order to expand and/or deploy the prosthetic valve 360. For instance, in the illustrated example, the handle 304 comprises first, second, and third knobs 306a, 306b, and 306c.

The first knob 306a can be a rotatable knob configured to produce axial movement of the outer shaft 308 relative to the prosthetic valve 360 in the distal and/or proximal directions in order deploy the prosthetic valve from the delivery sheath once the prosthetic valve has been advanced to a location at or adjacent the desired site of implantation within a patient. For instance, rotation of the first knob 306a in a first direction (e.g., clockwise) can retract the sheath proximally relative to the prosthetic valve 360 and rotation of the first knob 306a in a second direction (e.g., counterclockwise) can advance the sheath distally. In other configurations, the first knob 306a can actuated by sliding or moving the knob 306a axially, such as puling and/or pushing the knob. In still further configurations, actuations of the first knob 306a, such as by rotation or sliding the first knob 306a, can produce axial movement of the actuator assemblies 320 and thereby the prosthetic valve 360 relative to the delivery sheath to advance the prosthetic valve distally from the sheath.

The second knob 306b can be a rotatable knob configured to produce radial expansion and/or contraction of the prosthetic valve 360. For instance, rotation of the second knob 306b can move the actuator members and the support tubes 324 of actuator assemblies 320 axially relative to one another. The actuator members of assemblies 320 in turn cause corresponding movement of the actuators (e.g., actuators 170, 270 or 470) of the prosthetic valve. Rotation of the second knob 306b in a first direction (e.g., clockwise) can radially expand the prosthetic valve 360 and rotation of the second knob 306b in a second direction (e.g., counterclockwise) can radially collapse the prosthetic valve 360. In other configurations, the second knob 306b can be actuated by sliding or moving the knob 306b axially, such as pulling and/or pushing the knob.

The third knob 306c can be a rotatable knob configured to retain the prosthetic valve 360 in an expanded state. For instance, the third knob 306c can be operatively connected to a proximal end portion of the locking tool of each actuator assembly 320. Rotation of the third knob 306b in a first direction (e.g., clockwise) can rotate each locking tool to advance the locking nuts to their distal positions to resist radial compression of the frame of the prosthetic valve. Rotation of the knob 306c in the opposite direction (e.g., counterclockwise) can rotate each locking tool in the opposite direction to decouple each locking tool from the prosthetic valve 360. In other configurations, the third knob 306b can be actuated by sliding or moving the third knob 306b axially, such as pulling and/or pushing the knob. In some examples, the prosthetic valve can be self-locking, in which case a locking tool is not required. For example, the frame of the prosthetic valve can include locking features that automatically engage the actuator members of the prosthetic valve to resist radial compression of the prosthetic valve after it is expanded, such as disclosed in U.S. Application Nos. 63/085,947, 63/138,599, and 63/179,766.

Although not shown, the handle 304 can include a fourth rotatable knob operative connected to a proximal end portion of each actuator member. The fourth knob can be configured to rotate each actuator member, upon rotation of the knob, to unscrew each actuator member from the proximal portion of a respective actuator. As described above, once the locking tools and the actuator members are uncoupled from the prosthetic valve 360, they can be removed from the patient.

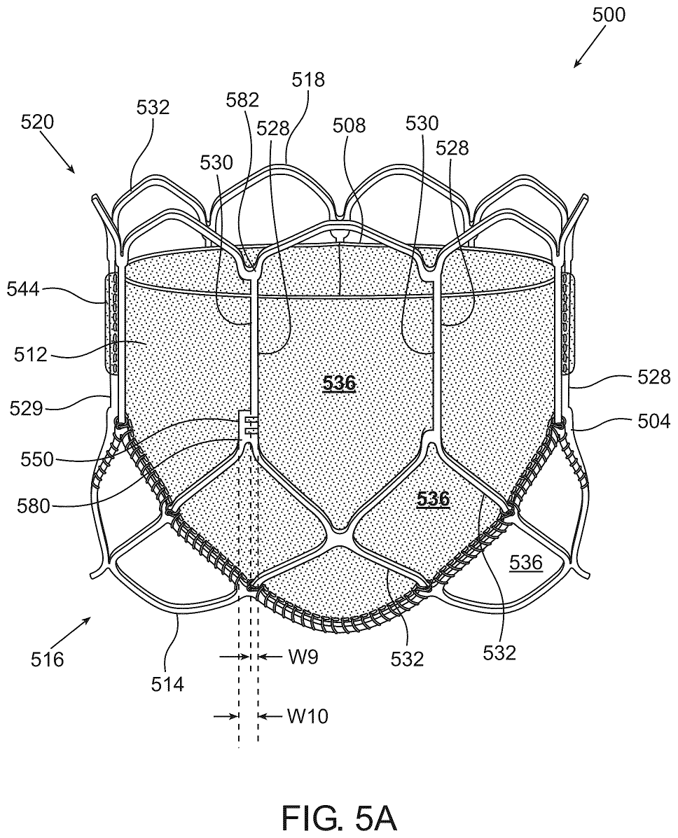

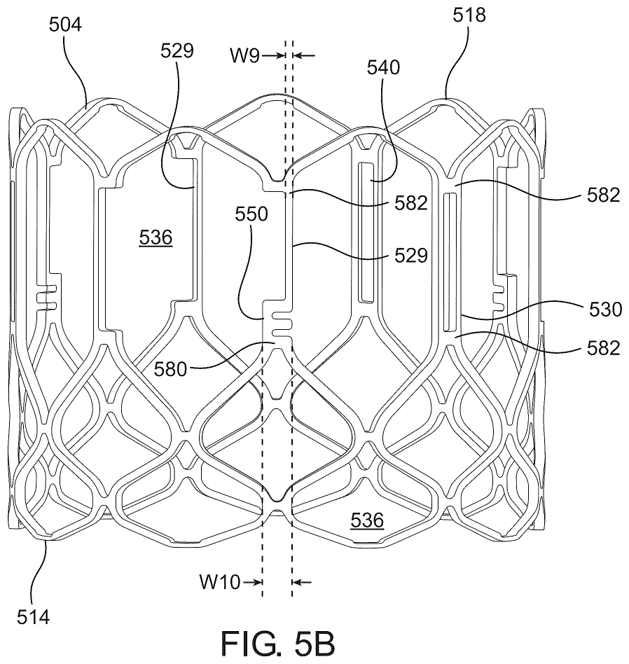

FIG. 5A-5B illustrate another example of a prosthetic valve 500. The prosthetic valve 500 can be configured to replace a native heart valve (e.g., aortic, mitral, pulmonary, and/or tricuspid valves). The prosthetic valve 500 is illustrated as a balloon expandable prosthetic valve movable between a radially compressed state and a radially expanded state. Balloon expandable valves generally involve a procedure of inflating a balloon within a prosthetic valve, thereby expanding the prosthetic valve 500 within the desired implantation site. The prosthetic valve 500 can include a frame 504 having an annular shape. The prosthetic valve 500 can further include a valvular structure 508 supported within and coupled to the frame 504.

The valvular structure 508 can include one or more leaflets 512, which can be identical to any example described above with respect to valvular structure 108 and leaflets 112, a detailed description of which is not repeated here in the interest of brevity. FIG. 5A shows the prosthetic valve 500 with the valvular structure 508, while FIG. 5B shows the frame 504 without the valvular structure.

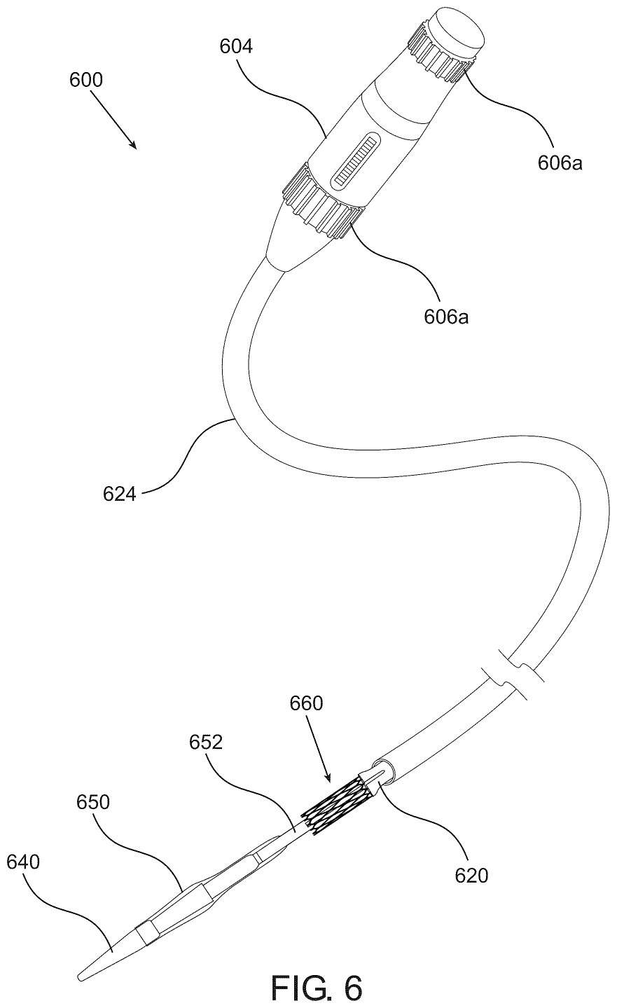

As further illustrated in FIG. 5A, the frame 504 has an inflow end 516, an outflow end 520, and a central longitudinal axis C extending in a direction from the inflow end 516 to the outflow end 520. The frame 504 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 504 can be crimped to a radially compressed state on a balloon catheter 652 (shown in FIG. 6), and then expanded inside a patient by an inflatable balloon. Alternatively or additionally, the frame 504 can be made of shape-memory materials such as, but not limited to, nickel titanium alloy (e.g., Nitinol).

Frame 504 comprises a plurality of intersecting struts, including curved or angled struts 532 and vertical struts 528. The frame 504 includes a plurality of strut rungs that can collectively define one or more rows of cells 536, wherein the vertical struts 528 in the illustrated example can be formed along the uppermost or proximal-most row of cells 536. The frame 504 can have a cylindrical or substantially cylindrical shape having a constant diameter from the inflow end 516 to the outflow end 520 as shown, or the frame can vary in diameter along the central longitudinal axis C, as disclosed in U.S. Pat. No. 9,155,619, which is incorporated herein by reference.

The end portions of angled struts 532 are forming outflow apices 518 at the outflow end 520 and inflow apices 514 at the inflow end 516. The struts can intersect at additional junctions formed between the outflow apices 518 and the inflow apices 514. The junctions can be equally or unequally spaced apart from each other, and/or from the apices 518, 514, between the outflow end 520 and the inflow end 516.

The vertical struts 528 can include commissure struts 529 and non-commissural vertical struts 530. The commissure struts 529 include commissure windows 540 that can accommodate commissures 544 of the valvular structure 508, while non-commissural vertical struts 530 are devoid of commissure windows. Each vertical strut 528, including any commissure strut 529 or non-commissural vertical strut 530, extends between a vertical strut inflow end 580 which is closer to the inflow end 516 of the valve 500, and a vertical strut outflow end 582 which is closer to the outflow end 520 of the valve 500. Two curved or angled struts 532 intersect with each vertical strut 528 at a vertical strut inflow end 580, which is circumferentially disposed between two adjacent inflow apices 514, and two curved or angled struts 532 intersect with each vertical strut 528 at a vertical strut outflow end 582, which is circumferentially disposed between two adjacent outflow apices 518.

Prosthetic valve 500 can further include an inner skirt (not shown) and/or an outer skirt (not illustrated in FIGS. 5A-5B, but can be generally similar to outer skirt 924 shown in FIG. 10), which can be implemented according to any example described for prosthetic valve 100, a detailed description of which is not repeated here in the interest of brevity.

The widths (in the circumferential direction) of non-commissural vertical strut, or of sidewalls at both sides of commissure windows in commissure struts, are usually to narrow to adequately accommodate internally formed information indicators therein. Thus, balloon expandable prosthetic valve disclosed in the current specification preferably include externally formed information indicators along portions of vertical struts thereof, the information indicators extending from the vertical strut inflow end or the vertical strut outflow end, to provide improved structural stability due to the angled or curved struts converging therewith, and are preferably devoid of internally formed information indicators.

FIGS. 5A-5B show a balloon expandable prosthetic valve 500 with non-commissural vertical struts 530 that include externally formed information indicators 550 extending from their vertical strut inflow ends 580. As shown, the non-commissural vertical strut 530 can have a width W9 in the circumferential direction, while the information indicator 550 formed therealong can have a width W10 in the circumferential direction, such that W10 is greater than W9 (W10>W9). In some implementations, a non-commissural vertical struts 530 which is narrower than the information indicator 550 can be aligned with one circumferential side of the information indicator 550, as shown in the illustrated example, or it can be aligned with a circumferential center of the information indicator 550 (not illustrated).

While the information indicator 550 is illustrated in FIGS. 5A-5B to be wider than the non-commissural vertical strut 530, in other implementations (not illustrated), the width of the externally formed information indicators 550 can be equal to the width of the non-commissural vertical struts 530 (i.e., W9=W10). Nevertheless, a wider information indicator 550 can advantageously serve as a support member to which other components can be attached. For example, in some implementations, a sensor (e.g., pressure sensor, flow sensor, temperature sensor, and the like) can be coupled to an information indicator 550.

While information indicator 550 is shown to extend from a vertical strut inflow end 580 of a non-commissural vertical strut 530 in the illustrated example, it is to be understood that an externally formed information indicator 550 can similarly extend from a vertical strut outflow end 582 of a non-commissural vertical strut 530. Similarly, while each non-commissural vertical strut 530 is shown in the illustrated example to include a single externally formed information indicator 550, in alternative implementations, a non-commissural vertical strut 530 can include two externally formed information indicators 550 at opposite ends thereof, one extending from its vertical strut inflow end 580, and the other extending from its vertical strut outflow end 582. Nevertheless, in some implementations, forming the information indicator 550 at the vertical strut inflow end 580 can be advantageous. As shown, the upper row of cells includes larger cells 536 compared with cells of other rows of the prosthetic valve. Frames designed with higher outflow cells 536 can result in more open space for blood flow and coronary access. In cases in which the width W10 of the information indicator 550 is greater than the width W9 of the non-commissural vertical strut 530, forming the wider information indicator 550 at the vertical strut inflow end 580 advantageously provides more open space (not blocked by the wider information indicator) at the outflow end of the frame for increased blood flow and coronary access.

While an E-shaped information indicator 550 is illustrated, it is to be understood that the externally formed information indicator 550 can be in the shape of any other alphanumeric character, symbol, or any other geometric shape. While a single externally formed information indicator 550 if shown to be formed along three non-commissural vertical strut 530 in the example illustrated in FIG. 5B, it is to be understood that any other number of non-commissural vertical strut 530 can include one or two externally formed information indicator 550. When a plurality of externally formed information indicators 550 are included, they can be similarly shaped, or differently shaped—for example to convey different information details of the prosthetic valve 500. In some implementations, one or more information indicators can further serve as support structures to which soft components of the prosthetic valve can be coupled. For example, skirts (such as an inner skirt and/or an outer skirt) and/or portions of leaflets of the prosthetic valve can be sutured to portions of one or more information indicator.

As mentioned above, since a support post, such as an internally formed non-commissural support post 526, may not provide sufficient space to include both digits, in some implementations, two adjacent support posts, such as two adjacent non-commissural support post 526, can each include one of the digits of a two-digit deployed diameter, which can be similar or different from each other. For example, for a prosthetic valve having a deployed diameter of 23 mm, one externally formed information indicator formed on a first non-commissural support post 526 can take the form of the first digit (e.g., “2”), while another externally formed information indicator formed on a second non-commissural support post 526, adjacent the first non-commissural support post 126, can take the form of the second digit (e.g., “3”).

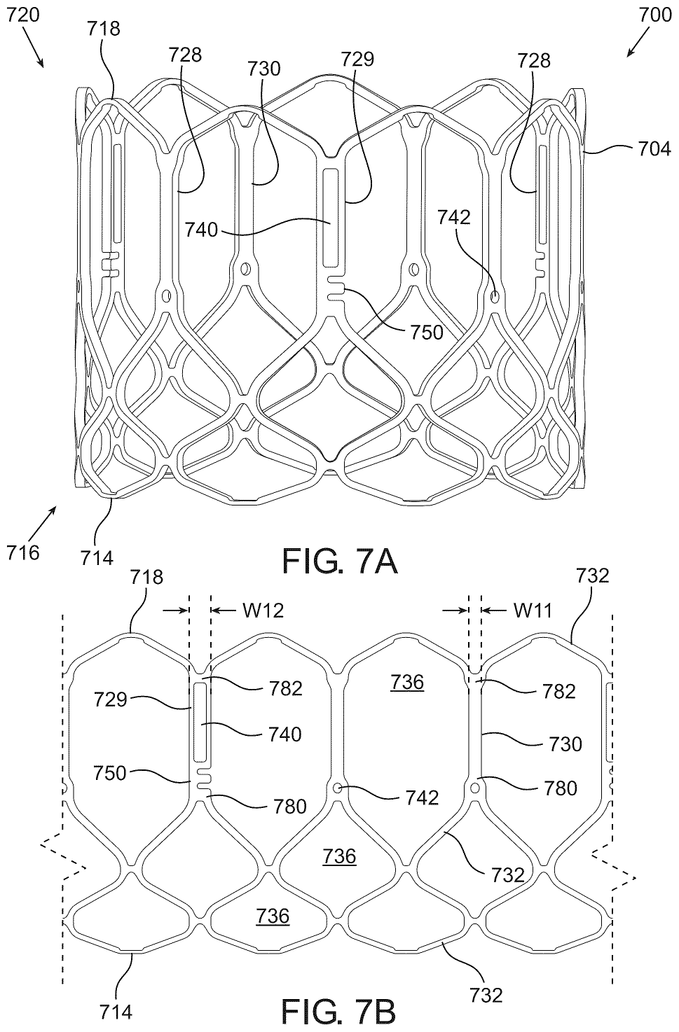

FIGS. 7A-7B illustrate another exemplary prosthetic valve 700, shown without soft components (such as skirts or the valvular structure) for clarity. Prosthetic valve 700 can be structurally similar to and function in a similar manner as the described prosthetic valve 500, except that prosthetic valve 700 is shown to have commissure struts 729 that include externally formed information indicator 750 extending from their respective vertical strut inflow ends 780. For instance, the prosthetic valve 700 can include a frame 704 extending between an inflow end 716 and an outflow end 720, the frame 704 including a plurality of vertical struts 728 that include commissure struts 729 equipped with commissure windows 740, and non-commissural vertical struts 730 devoid of commissure windows. The frame 704 can also include a plurality of curved or angled struts 732 intersecting with other angled struts 732 and interconnecting the vertical struts 728, wherein the struts 732 and the vertical struts 728 define cells 736 of the frame 704. FIG. 7A shows an annular configuration of the frame 704, while FIG. 7B shows a portion of a flattened configuration of the frame 704 for illustrative purpose.

Each vertical strut 728, including any commissure strut 729 or non-commissural vertical strut 730, extends between a vertical strut inflow end 780 which is closer to the inflow end 716 of the valve 700, and a vertical strut outflow end 782 which is closer to the outflow end 720 of the valve 700. Two curved or angled struts 732 intersect with each vertical strut 728 at a vertical strut inflow end 780, which is circumferentially disposed between two adjacent inflow apices 714, and two curved or angled struts 732 intersect with each vertical strut 728 at a vertical strut outflow end 782, which is circumferentially disposed between two adjacent outflow apices 718. Other structural and functional components of the prosthetic valve 700 can be similar to those described above with respect to prosthetic valve 500, with like numbers referring to like components, and in the interest of brevity will not be further described.

As shown in FIGS. 7A-7B, both the commissure strut 729 and the information indicator 550 can have a common equal width W12 in the circumferential direction. In other implementations, the width of the information indicator 550 can be greater than that of the commissure strut 729. The non-commissural vertical strut 730 can have a width W11 in the circumferential direction, which can be similar to the width of the commissure strut 729, or narrower than the width of the commissure strut 729, since the non-commissural vertical strut 730 does not include a commissure window 740.

In some implementations, as shown for frame 704, one or more of the non-commissural vertical struts 730 can have a vertical strut inflow end 780 that is widened relative to its middle portion (relative to W11). In some implementations, the vertical strut inflow end 780 of a non-commissural vertical struts 730 can comprise an aperture 742. The apertures 742 can be configured to receive fasteners (e.g., sutures) for attaching soft components of the prosthetic valve 700 to the frame 704. For example, in some implementations, an outer skirt can be positioned around an outer surface of the frame 704 and secured to the apertures 742. While non-commissural vertical struts 730 comprising widened vertical strut inflow ends 780 with apertures 742 are illustrated and described with respect to prosthetic valve 700, it is to be understood that prosthetic valve 500, as well as any other balloon expandable prosthetic valve disclosed herein, can include at least one non-commissural vertical strut comprising a widened vertical strut inflow end with an aperture.

While an E-shaped information indicator 750 is illustrated, it is to be understood that the information indicator 750 can be in the shape of any other alphanumeric character, symbol, or any other geometric shape. While a single information indicator 750 if shown to be formed along all three commissure struts 729 in the example illustrated in FIG. 7A, it is to be understood that any other number of commissure struts 729 can include an externally formed information indicator 750.

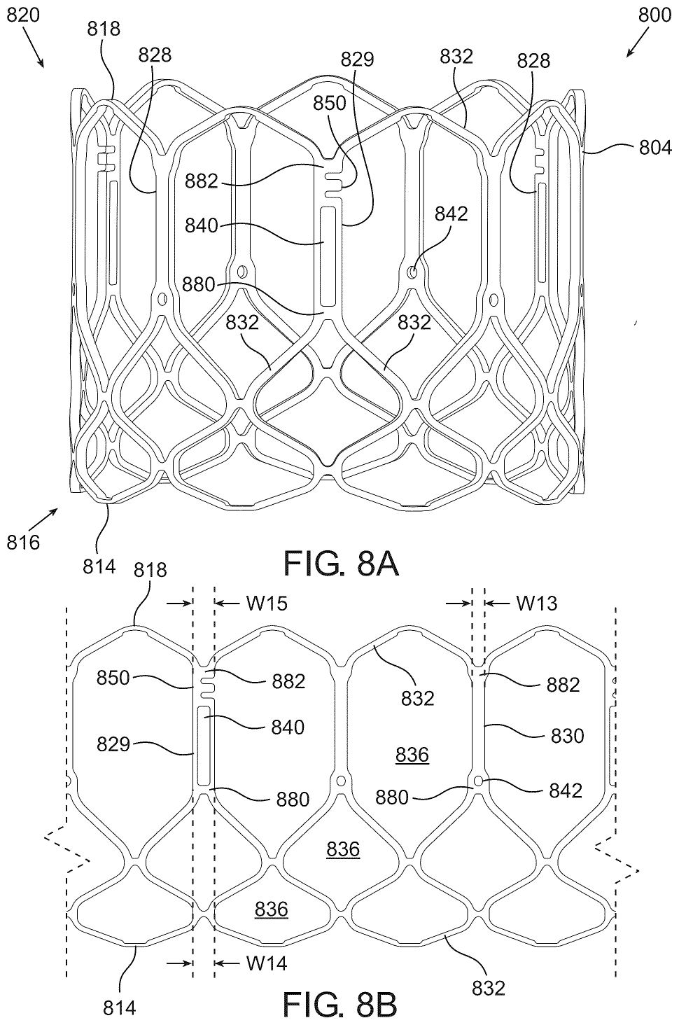

FIGS. 8A-8B illustrate another exemplary prosthetic valve 800, shown without soft components (such as skirts or the valvular structure) for clarity. Prosthetic valve 800 can be structurally similar to and function in a similar manner as the described prosthetic valve 500 or 700, except that prosthetic valve 800 is shown to have commissure struts 829 that include externally formed information indicator 850 extending from their respective vertical strut outflow ends 880. For instance, the prosthetic valve 800 can include a frame 804 extending between an inflow end 816 and an outflow end 820, the frame 804 including a plurality of vertical struts 828 that include commissure struts 829 equipped with commissure windows 840, and non-commissural vertical struts 830 devoid of commissure windows. The frame 804 can also include a plurality of curved or angled struts 832 intersecting with other angled struts 832 and interconnecting the vertical struts 828, wherein the struts 832 and the vertical struts 828 define cells 836 of the frame 804. FIG. 8A shows an annular configuration of the frame 804, while FIG. 8B shows a portion of a flattened configuration of the frame 804 for illustrative purpose.

Each vertical strut 828, including any commissure strut 829 or non-commissural vertical strut 830, extends between a vertical strut inflow end 880 which is closer to the inflow end 816 of the valve 800, and a vertical strut outflow end 882 which is closer to the outflow end 820 of the valve 800. Two curved or angled struts 832 intersect with each vertical strut 828 at a vertical strut inflow end 880, which is circumferentially disposed between two adjacent inflow apices 814, and two curved or angled struts 832 intersect with each vertical strut 828 at a vertical strut outflow end 882, which is circumferentially disposed between two adjacent outflow apices 818. Other structural and functional components of the prosthetic valve 800 can be similar to those described above with respect to prosthetic valve 500 or 700, with like numbers referring to like components, and in the interest of brevity will not be further described.

As shown in FIGS. 8A-8B, the commissure strut 829 can have a width W14 in the circumferential direction, and the externally formed information indicator 850 can have a width W15 in the circumferential direction, such that W15 can be substantially equal to W14 (W15=W14) as shown in the illustrated example, or W15 can be greater than W14 (W15>W14). The non-commissural vertical strut 830 can have a width W13 in the circumferential direction, which can be similar to the width W14 of the commissure strut 829, or narrower than the width W14, since the non-commissural vertical strut 830 does not include a commissure window 840. As further shown, at least one non-commissural vertical struts 830 can optionally include a widened vertical strut inflow ends 880 with aperture 842.

While an E-shaped information indicator 850 is illustrated, it is to be understood that the information indicator 850 can be in the shape of any other alphanumeric character, symbol, or any other geometric shape. While a single information indicator 850 is shown to be formed along all three commissure struts 829 in the example illustrated in FIG. 8A, it is to be understood that any other number of commissure struts 829 can include an externally formed information indicator 850.

While commissure struts 729 with information indicators 750 extending from their vertical strut inflow ends 780 are illustrated in FIGS. 7A-7B, and commissure struts 829 with information indicators 850 extending from their vertical strut outflow ends 882 are illustrated in FIGS. 8A-8B, it is to be understood that these features can be combined, such that one or more commissure strut will include an externally formed information indicator extending from its vertical strut inflow end, while one or more other commissure strut will include an externally formed information indicator extending from its vertical strut outflow end. Moreover, any such frame can include, in some implementations, one or more commissure strut equipped with two information indicators disposed at both sides thereof, one of which extends from its vertical strut inflow end, while the other extends from its vertical strut outflow end. Furthermore, a frame of a balloon expendable valve can include a combination of one or more commissure strut having one or two externally formed information indicators formed therealong, and at least one non-commissural vertical strut having one or two externally formed information indicators formed therealong. When a plurality of externally formed information indicators are included, they can be similarly shaped, or differently shaped.

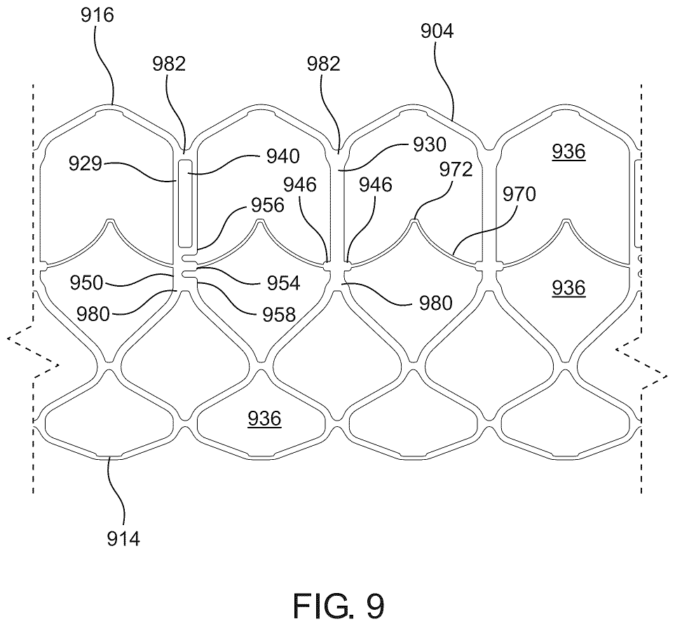

FIGS. 9-10 illustrate another exemplary prosthetic valve 900. Prosthetic valve 900 can be structurally similar to and function in a similar manner as the described prosthetic valve 700, except that prosthetic valve 900 further comprises support struts 970, as will be explained in greater detail below. For instance, the prosthetic valve 900 can include a frame 904 extending between an inflow end 916 and an outflow end 920, the frame 904 including a plurality of vertical struts 928 that include commissure struts 929 equipped with commissure windows 940, and non-commissural vertical struts 930 devoid of commissure windows. The frame 904 can also include a plurality of curved or angled struts 932 intersecting with other angled struts 932 and interconnecting the vertical struts 928, wherein the struts 932 and the vertical struts 928 define cells 936 of the frame 904. FIG. 9 shows a portion of a flattened configuration of the frame 904, while FIG. 10 shows an annular configuration of the prosthetic valve 900, including a valvular structure 908 with leaflets 912 and an outer skirt 924 disposed around the frame 904.

Each vertical strut 928, including any commissure strut 929 or non-commissural vertical strut 930, extends between a vertical strut inflow end 980 which is closer to the inflow end 916 of the valve 900, and a vertical strut outflow end 982 which is closer to the outflow end 920 of the valve 900. Two curved or angled struts 932 intersect with each vertical strut 928 at a vertical strut inflow end 980, which is circumferentially disposed between two adjacent inflow apices 914, and two curved or angled struts 932 intersect with each vertical strut 928 at a vertical strut outflow end 982, which is circumferentially disposed between two adjacent outflow apices 918. Other structural and functional components of the prosthetic valve 900 can be similar to those described above with respect to prosthetic valve 700, with like numbers referring to like components, and in the interest of brevity will not be further described.

As shown in FIG. 9, the commissure struts 929 include externally formed information indicator 950 extending from their respective vertical strut outflow ends 980, distal to the commissure windows 940. While each commissure strut 929 is shown to include a single information indicator 950 extending from the vertical strut outflow ends 980, in some implementations, at least one commissure strut 929 can include two externally formed information indicators 950 at opposite ends thereof, one extending from its vertical strut inflow end 980, and the other extending from its vertical strut outflow end 982. Moreover, while all non-commissural vertical struts 930 are shown without any information indicators, in some implementations, at least one non-commissural vertical strut 930 of the frame 904 can further include one or two externally formed information indicators 950 as well.

FIG. 10 illustrates an outer skirt 924 disposed around and attached to an outer surface of the frame 904. The outer skirt 924 can be secured to the frame 904 by one or more sutures 926 that can looped around angled struts 932 of the frame 904. While an inflow end portion of the outer skirt 924 can be sutured to angled struts 932 along the inflow end 916 of the prosthetic valve 900, the outflow end 922 of the outer skirt 924 is disposed along the upper or proximal-most row of cells 936, which are generally axially longer than other cells 936 of the frame 904, as these cells include the vertical struts 928, creating large openings defines by the cells 936 of the upper row. Such enlarged cell openings create voids that cannot provide adequate support for the outflow end 922 of the outer skirt 924, which may result in the portions of the outer skirt 924 bulging somewhat radially inward through these cell openings, or may limit the total height of the outer skirt 924 to terminate at its outflow end 922 below the opening of the uppermost cells 936.

As shown in FIG. 9, the frame 904 further includes support struts 970, which can be curved or angled support struts, such that at least two support struts 970 extend between at least two adjacent vertical struts 928 and intersect with each other at a support apex 972, which can be axially aligned with a corresponding outflow apex 918, and wherein at least one support strut 370 extends from an externally formed information indicator 950.

Some shapes of an externally formed information indicator, such as an E-shaped or a 3-shaped information indicator, can include portions that can be advantageously utilized as a structural base member from which a support strut can extend in a stable manner. For example, an E-shaped externally formed information indicator 950 shown in FIG. 9 can include an inflow portion 958 at an end thereof coinciding with the vertical strut inflow end 980 connected to two angled struts 932, an outflow portion 956 at an opposite end thereof that is closer to the commissure window 940, and a central portion 954 disposed therebetween, in the form of a laterally (or circumferentially) oriented extension.

As shown, at least one support strut 970 can extend from the central portion 954, and intersect with another support strut 970 extending from a neighboring non-commissural vertical strut 930. In some implementations, the non-commissural vertical strut 930 comprises a short lateral extension 946 from which the corresponding support strut 970 can extend. As shown, if two support struts 970 extend in opposite directions from the same non-commissural vertical strut 930, the non-commissural vertical strut 930 can comprise two lateral extensions 946 oriented in opposite lateral directions (perpendicular to the axial direction of the vertical strut 930) to support both support struts 970 extending therefrom. The lateral extension 946 can be axially aligned with a corresponding central portion 954 of the information indicator 950.

Preferably, the frame 904 includes two support strut 970 intersecting with each other between each two adjacent vertical struts 928 along the upper or proximal-most row of cells 936. As shown in FIG. 10, the outflow end 922 of the outer skirt 924 can be attached, for example via sutures 926, to support struts 970. As further shown in FIG. 9, since the support struts 970 mainly serve to support outer skirt attachment, they do not need to withstand significant loads or experience the same stress levels as other struts 932 of the frame 904, and can thus be relatively narrower and/or thinner than other angled struts 932, thus avoiding risk of significantly increasing a crimped profile of the prosthetic valve 900 during delivery.

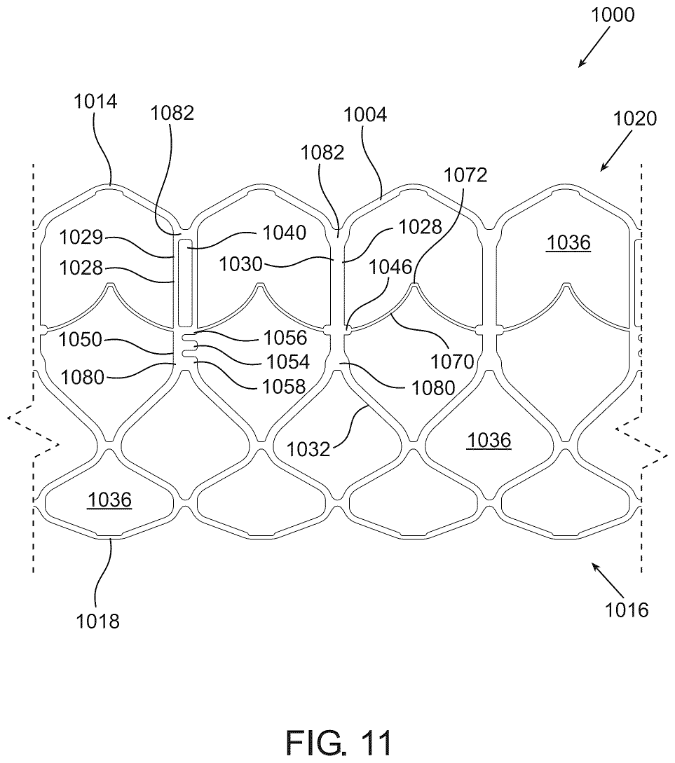

FIG. 11 shows a portion of a flattened configuration of a frame 1004 of another exemplary prosthetic valve 1000. Prosthetic valve 1000 can be structurally similar to and function in a similar manner as the described prosthetic valve 900, except that at least one support strut 1070 extends from an outflow portion 1056 of an externally formed information indicator 1050 instead of its central portion. For instance, the prosthetic valve 1000 includes a frame 1004 extending between an inflow end 1016 and an outflow end 1020, the frame 1004 including a plurality of vertical struts 1028 that include commissure struts 1029 equipped with commissure windows 1040, and non-commissural vertical struts 1030 devoid of commissure windows. The frame 1004 can also include a plurality of curved or angled struts 1032 intersecting with other angled struts 1032 and interconnecting the vertical struts 1028, wherein the struts 1032 and the vertical struts 1028 define cells 1036 of the frame 1004.

Each vertical strut 1028, including any commissure strut 1029 or non-commissural vertical strut 1030, extends between a vertical strut inflow end 1080 which is closer to the inflow end 1016 of the valve 1000, and a vertical strut outflow end 1082 which is closer to the outflow end 1020 of the valve 1000. Two curved or angled struts 1032 intersect with each vertical strut 1028 at a vertical strut inflow end 1080, which is circumferentially disposed between two adjacent inflow apices 1014, and two curved or angled struts 1032 intersect with each vertical strut 1028 at a vertical strut outflow end 1082, which is circumferentially disposed between two adjacent outflow apices 1018.

The commissure struts 1029 include externally formed information indicators 1050 extending from their respective vertical strut outflow ends 1080, distal to the commissure windows 1040. The frame 1004 further includes support struts 1070, which can be curved or angled support struts, such that at least two support struts 1070 extend between at least two adjacent vertical struts 1028 and intersect with each other at a support apex 1072, which can be axially aligned with a corresponding outflow apex 1018, and wherein at least one support strut 1070 extends from an externally formed information indicator 1050. The information indicator 1050, which is illustrated as an E-shaped indicator in the illustrated example (but can have other shapes in other examples), can include an inflow portion 1058 at an end thereof connected to two angled struts 1032, an outflow portion 1056 at an opposite end thereof that is closer to the commissure window 1040, and a central portion 1054 disposed therebetween, in the form of a laterally (or circumferentially) oriented extension. Other structural and functional components of the prosthetic valve 1000 can be similar to those described above with respect to prosthetic valve 900, with like numbers referring to like components, and in the interest of brevity will not be further described.

As mentioned above and shown in FIG. 11, at least one support strut 1070 can extend from the outflow portion 1056, and intersect with another support strut 1070 extending from a neighboring non-commissural vertical strut 1030. In some implementations, the non-commissural vertical strut 1030 comprises a short lateral extension 1046 from which the corresponding support strut 1070 can extend. As shown, if two support struts 1070 extend in opposite directions from the same non-commissural vertical strut 1030, the non-commissural vertical strut 1030 can comprise two lateral extensions 1046 oriented in opposite lateral directions (perpendicular to the axial direction of the vertical strut 1030) to support both support struts 1070 extending therefrom. The lateral extension 1046 can be axially aligned with a corresponding outflow portion 1056 of the information indicator 1050. The configuration shown in FIG. 11 can be advantageous over that shown in FIG. 9 in that the outflow portion 1056, which can also serve as, to continuously extend to form a part of, the inflow end of the commissure window 1040, will provide a more rigid and stable support to the support strut 1070 extending therefrom, compared to the central portion 1054, for example.

Preferably, the frame 1004 includes two support strut 1070 intersecting with each other between each two adjacent vertical struts 1028 along the upper or proximal-most row of cells 1036. The outflow end portion of an outer skirt can be attached to support struts 1070 in the same manner described above with respect to FIG. 10. As further shown in FIG. 11, since the support struts 1070 mainly serve to support outer skirt attachment, they do not need to withstand significant loads or experience the same stress levels as other struts 1032 of the frame 1004, and can thus be relatively narrower and/or thinner than other angled struts 1032, thus avoiding risk of significantly increasing a crimped profile of the prosthetic valve 1000 during delivery.

While each commissure strut 1029 is shown to include a single information indicator 1050 extending from the vertical strut outflow ends 1080, in some implementations, at least one commissure strut 1029 can include two externally formed information indicators 1050 at opposite ends thereof, one extending from its vertical strut inflow end 1080, and the other extending from its vertical strut outflow end 1082. Moreover, while all non-commissural vertical struts 1030 are shown without any information indicators, in some implementations, at least one non-commissural vertical strut 1030 of the frame 1004 can further include one or two externally formed information indicators 1050 as well.

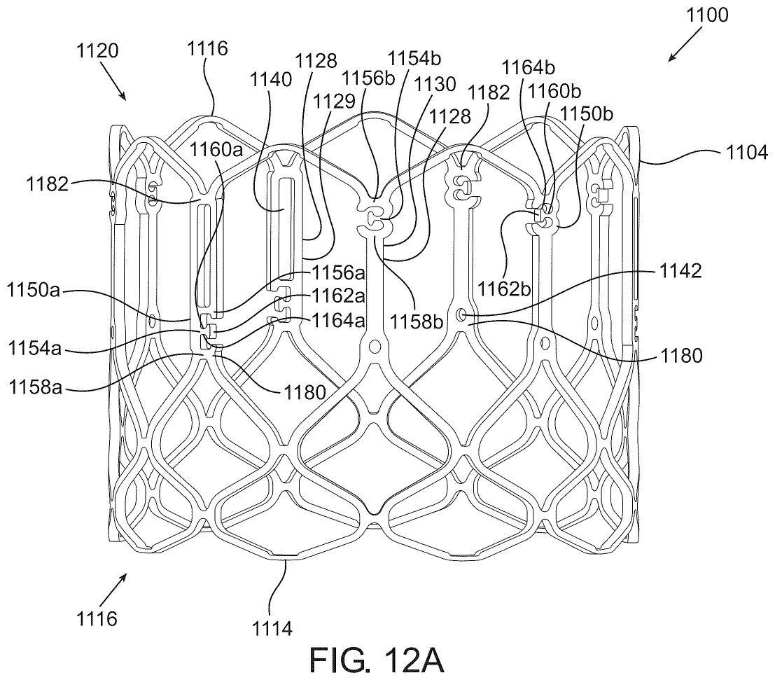

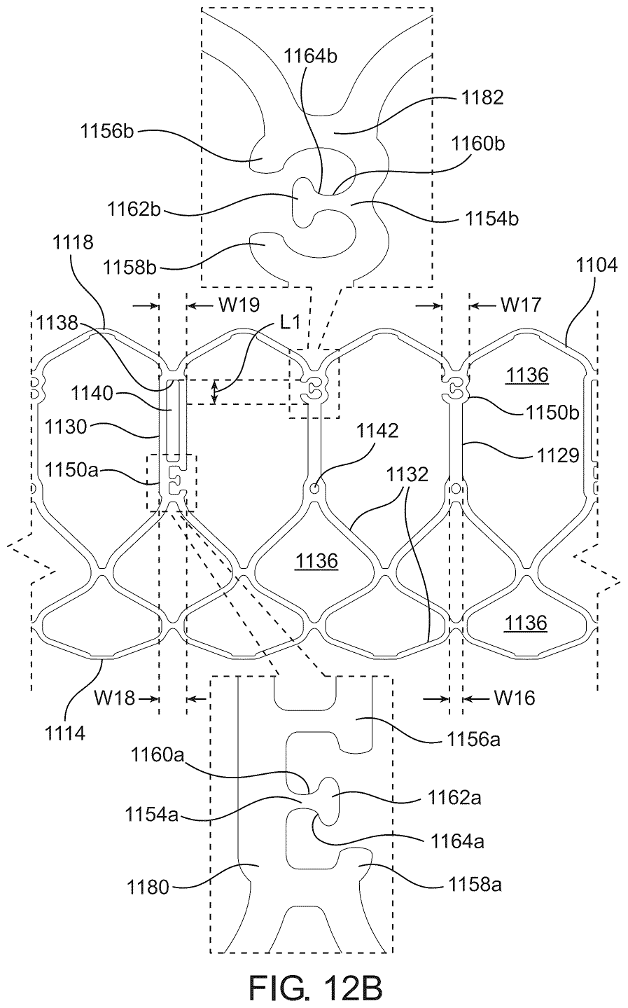

FIGS. 12A-12B illustrate another exemplary prosthetic valve 1100, shown without soft components (such as skirts or the valvular structure) for clarity. Prosthetic valve 1100 can be structurally similar to and function in a similar manner as the described prosthetic valve 700, except for the features that will be described below. For instance, the prosthetic valve 1100 can include a frame 1104 extending between an inflow end 1116 and an outflow end 1120, the frame 1104 including a plurality of vertical struts 1128 that include commissure struts 1129 equipped with commissure windows 1140, and non-commissural vertical struts 1130 devoid of commissure windows. The frame 1104 can also include a plurality of curved or angled struts 1132 intersecting with other angled struts 1132 and interconnecting the vertical struts 1128, wherein the struts 1132 and the vertical struts 1128 define cells 1136 of the frame 1104. FIG. 12A shows an annular configuration of the frame 1104, while FIG. 12B shows a portion of a flattened configuration of the frame 1104 for illustrative purpose.

Each vertical strut 1128, including any commissure strut 1129 or non-commissural vertical strut 1130, extends between a vertical strut inflow end 1180 which is closer to the inflow end 1116 of the valve 1100, and a vertical strut outflow end 1182 which is closer to the outflow end 1120 of the valve 1100. Two curved or angled struts 1132 intersect with each vertical strut 1128 at a vertical strut inflow end 1180, which is circumferentially disposed between two adjacent inflow apices 1114, and two curved or angled struts 1132 intersect with each vertical strut 1128 at a vertical strut outflow end 1182, which is circumferentially disposed between two adjacent outflow apices 1118. Other structural and functional components of the prosthetic valve 1100 can be similar to those described above with respect to prosthetic valve 700, with like numbers referring to like components, and in the interest of brevity will not be further described.

In the illustrated example, the frame 1104 is shown to include two types of differently formed information indicators 1150, such as E-shaped information indicators 1150a extending from the inflow portions 1180 of commissure struts 1129, and 3-shaped information indicators 1150b extending from the outflow portions 1182 of non-commissural vertical struts 1130. However, it is to be understood that this combination is shown by way of illustration and not limitation, and that the frame 1104 can include a more or less information indicators 1150 similarly or differently formed, wherein each externally formed information indicators 1150 can be in the shape of any other alphanumeric character, symbol, or any other geometric shape.

While information indicators 1150b are shown to be formed along all six non-commissural vertical struts 1130 in the example illustrated in FIG. 12A, it is to be understood that any other number of non-commissural vertical struts 1130 can include externally formed information indicators 1150b. Similarly, while information indicators 1150a are shown to be formed along all three commissure struts 1129 in the example illustrated in FIG. 12A, it is to be understood that any other number of commissure struts 1129 can include externally formed information indicators 1150a.

While information indicator 1150a is shown to extend from a vertical strut inflow end 1180 of a commissure strut 1129 in the illustrated example, it is to be understood that an externally formed information indicator 1150a can similarly extend from a vertical strut outflow end 1182 of a commissure strut 1129. Similarly, while each commissure strut 1129 is shown in the illustrated example to include a single externally formed information indicator 1150a, in alternative implementations, a commissure strut 1129 can include two externally formed information indicators 1150a at opposite ends thereof, one extending from its vertical strut inflow end 1180, and the other extending from its vertical strut outflow end 1182.

While information indicator 1150b is shown to extend from a vertical strut outflow end 1182 of a non-commissural vertical strut 1130 in the illustrated example, it is to be understood that an externally formed information indicator 1150b can similarly extend from a vertical strut inflow end 1180 of a non-commissural vertical strut 1130. Similarly, while each non-commissural vertical strut 1130 is shown in the illustrated example to include a single externally formed information indicator 1150b, in alternative implementations, a non-commissural vertical strut 1130 can include two externally formed information indicators 1150b at opposite ends thereof, one extending from its vertical strut inflow end 1180, and the other extending from its vertical strut outflow end 1182.

As shown in FIG. 12B, the non-commissural vertical strut 1130 can have a width W16 in the circumferential direction, and an externally formed information indicator 1150b formed therealong, can have a width W17 in the circumferential direction, such that W17 is greater than W16 (W17>W16). The commissure strut 1129 can have a width W19 in the circumferential direction, and an externally formed information indicator 1150a formed therealong can have a width W18 in the circumferential direction, such that W18 can be substantially equal to W19 (W18=W19) as shown in the illustrated example, or W18 can be greater than W19 (W18>W19). The width W19 of the commissure strut 1129 can be equal to the width W16 of the non-commissural vertical strut 1130, or as illustrated, W19 can be greater than W16, since the non-commissural vertical strut 1130 does not include a commissure window 1140. As further shown, at least one non-commissural vertical struts 1130 can optionally include a widened vertical strut inflow ends 1180 with aperture 1142.

In some examples, externally formed information indicators 1150b formed along non-commissural vertical struts 1130 can protect the leaflets during crimping. When a prosthetic valve 1100 is placed in a crimping apparatus to radially compress the valve to a smaller diameter for insertion into a patient, the leaflets of the valve are pressed against the inner surface of the metal frame 1104 and portions of the tissue (for example, in the case of leaflets made of tissue material) can protrude into the open cells of the frame between the struts and can be pinched due to the scissor-like motion of the struts of the frame. If the valve is severely crimped to achieve a small crimping size, this scissor-like motion can result in cuts and rupture of the tissue leaflets.

In the illustrated example, the wider information indicators 1150b can have a width designed to limit the crimped size so as to maintain a minimal space, in the circumferential direction, between adjacent vertical struts 1128. Specifically, when a prosthetic valve 1100 is crimped, adjacent vertical struts move closer to each other, an may pinch leaflet material protruding through the cells openings therebetween. However, approximation of vertical struts 1128 toward each other will halt when information indicators 1150 contact adjacent information indicators 1150 or adjacent vertical struts 1128. Specifically, compression of the prosthetic valve 1100 will halt when an externally formed information indicator 1150b of a non-commissural vertical strut 1130 will contact another information indicator 1150b of an adjacent non-commissural vertical strut 1130, or when it will contact an adjacent commissure strut 1129.

If crimping is stopped when two adjacent information indicators 1150b of two adjacent non-commissural vertical struts 1130 contact each other, the resulting minimal lateral space between the adjacent non-commissural vertical struts 1130 will be equal to the difference between the widths W17 and W16 (i.e., W17−W16). If crimping is stopped when an information indicator 1150 contact an adjacent commissure strut 1129, the resulting minimal lateral space between the adjacent non-commissural vertical strut 1130 and the commissure strut 1129 will be equal to half the difference between the widths W17 and W16 (i.e., [W17−W16]/2). If one fold of a leaflet extends through such a space formed between the adjacent vertical struts 1128, it may be desired for this space to be larger enough to accommodate both layers of the folded portion overlaying each other. Thus, twice the thickness T1 of a leaflet should be extendable between adjacent vertical struts 1128 without being forcibly pinched. In some implementations, the difference (W17−W16) is at least as great as twice the thickness T1 of a leaflet. In some implementations, half of this difference (i.e., [W17−W16]/2) is at least as great as twice the thickness T1 of a leaflet, meaning that the difference (W17−W16) is at least four times as great as the thickness T1 of a leaflet. In some cases, the tissue material of a leaflet (e.g., pericardium) can be squeezed up to about at least half of its free (i.e., un-squeezed) thickness, without inflicting long-term damage to the tissue material. In such cases, the difference (W17−W16) can be at least as great the thickness T1 of a leaflet (if the leaflet extends between two adjacent non-commissural vertical struts 1130), or at least as great as twice the thickness T1 of a leaflet (if the leaflet extends between a non-commissural vertical strut 1130 and a commissure strut 1129).

While optional interaction between adjacent information indicators 1150b of two adjacent non-commissural vertical struts 1130, or between an information indicator 1150b of a non-commissural vertical strut 1130 and an adjacent commissure strut 1129 is described above, for commissure struts that do not include an information indicator 1150 at the same axial position as that of the information indicator 1150b of the non-commissural vertical strut 1130, it is to be understood that in alternative implementations, crimping can be stopped when an information indicator 1150b of a non-commissural vertical strut 1130 contacts an adjacent information indicator 1150a formed at the same axial position of a commissure strut 1129. For example, a commissure strut 1129 can include an information indicator 1150a extending from its vertical strut outflow end 1182, configured to approximate up to full contact, during crimping, an adjacent information indicator 1150b extending from the vertical strut outflow end 1182 of a neighboring non-commissural vertical strut 1130. This can be advantageous if the information indicator 1150a of the commissure strut 1129 has a width W18 that is greater than the width W19 of the commissure strut 1129.

When information indicators 1150 are further utilized as stoppers that limit the minimal crimping diameter of the prosthetic valve 1000 as described above, it may be preferable for them to extend from vertical strut outflow ends 1182, as shown for information indicators 1150b in the illustrated example, disposed at an axial height which is proximal to the leaflets, to prevent such leaflets from being accidentally pinched by the information indicators 1150 themselves. A frame of a prosthetic valve that includes interconnected angled struts, such as frame 1104, foreshortens during expansion, such that it's length in an expanded state, designated H1, is less than its length in a crimped state, designated H2 (i.e., H1<H2). Leaflets of the valvular structure can include relatively high tabs extending through the commissure windows to form the commissures, such that the outflow ends of the remainder of the leaflets can extend inwardly in the radial direction and somewhat distally in the axial direction from the respective commissures 1144. Since the frame 1104 elongates during crimping, the outflow portions of the leaflets can extend further in the proximal direction in a crimped state of the prosthetic valve.

In order to mitigate the risk of the leaflet material being pinched between an information indicator 1150b extending from the vertical strut outflow end 1182 of a non-commissural vertical strut 1130, and the sidewalls of a commissure window 1140 of an adjacent commissure strut 1129, a minimal distance L1 can be defined between the outflow edge 1138 of the commissure window 1140, and the lower end of the information indicator 1150b, which can be defined by its inflow portion 1158. Thus, L1 can be defined as the outflow edge 1138 of the commissure window and the inflow portion 1158 of an information indicators 1150b extending from the vertical strut outflow end 1182 of an adjacent non-commissural vertical strut 1130. In some implementation, the distance L1 is less than the difference in height of the frame 1104 between its crimped and expanded states (i.e., L1<H2−H1). Axial height H1 or H2 is measured between the inflow end 1116 and the outflow end 1120.

As described above with respect to externally formed information indicators 950 and 1050, some information indicators can be shaped to include a central portion in the form of a laterally oriented, free ended, extension. Two such shapes are illustrated in zoomed in sections of FIG. 12B, namely an E-shaped information indicator 1150a and a 3-shaped information indicator 1150b, each of which includes an inflow portion 1158 at an end connected to two angled struts 1132, an outflow portion 1156 at an opposite end thereof, and a central portion 1154 disposed therebetween, in the form of a laterally oriented extension.

The central portion 1154 can be further utilized, in some implementation, as a structural element to which soft components of the prosthetic valve 1100, such as an inner skirt, an outer skirt, or a leaflet, can be attached, such as by being sutured thereto. However, if the central portion is formed as a free-ended extension having a uniform width (defined between the proximal and distal edges of the central portion), a suture looped thereover or tied thereto may slip from its free end. In order to prevent a suture from slipping from the central portion of an information indicator, the central portion can further include, in some implementations, a lateral body 1160 extending in a lateral direction (for example, substantially parallel to the outflow portion 1156 and/or inflow portion 1158) and terminating at a bi-directional vertical extension 1162 at the free end of the central portion 1154. The bi-directional vertical extension 1162 can be perpendicular to the lateral body 1160, extending in both the proximal and distal directions from the free end of the central portion 1154. This creates two shoulders 1164 formed at the transition from the lateral body 1160 to the bi-directional vertical extension 1162, which can serve to better retain a suture attached to the central portion 1154 and prevent it from slipping therefrom.

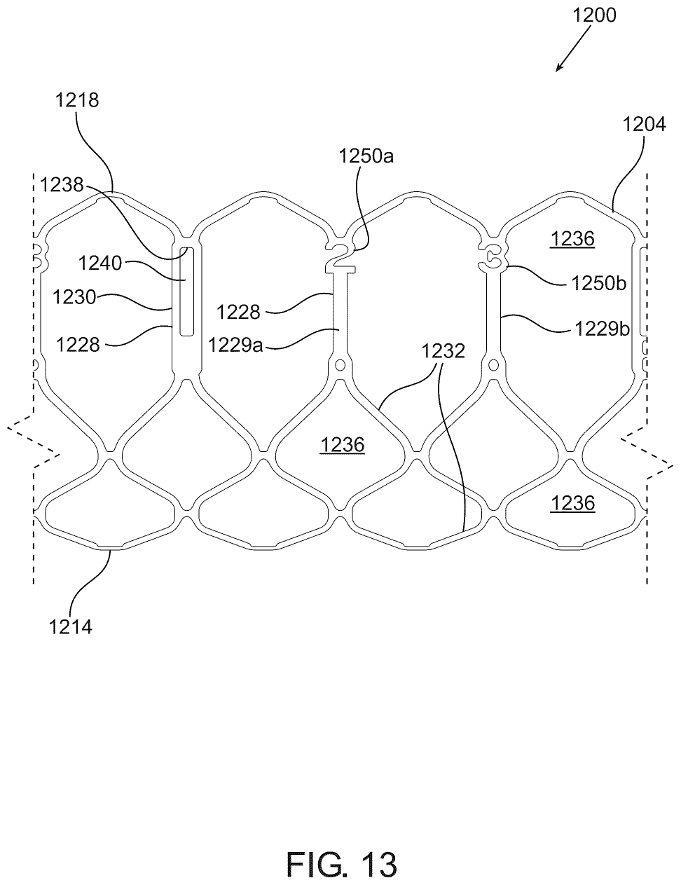

FIG. 13 shows a portion of a flattened configuration of a frame 1204 of another exemplary prosthetic valve 1200. Prosthetic valve 1200 can be structurally similar to and function in a similar manner as any of the prosthetic valves 500, 500, 700, 800, 900, 1000 and 1100 described above. For instance, the prosthetic valve 1200 includes a frame 1204 extending between an inflow end 1216 and an outflow end 1220, the frame 1204 including a plurality of vertical struts 1228 that include commissure struts 1229 equipped with commissure windows 1240, and non-commissural vertical struts 1230 devoid of commissure windows. The frame 1204 can also include a plurality of curved or angled struts 1232 intersecting with other angled struts 1232 and interconnecting the vertical struts 1228, wherein the struts 1232 and the vertical struts 1228 define cells 1236 of the frame 1204.

Each vertical strut 1228, including any commissure strut 1229 or non-commissural vertical strut 1230, extends between a vertical strut inflow end 1280 which is closer to the inflow end 1216 of the valve 1200, and a vertical strut outflow end 1282 which is closer to the outflow end 1220 of the valve 1200. Two curved or angled struts 1232 intersect with each vertical strut 1228 at a vertical strut inflow end 1280, which is circumferentially disposed between two adjacent inflow apices 1214, and two curved or angled struts 1232 intersect with each vertical strut 1228 at a vertical strut outflow end 1282, which is circumferentially disposed between two adjacent outflow apices 1218. Other structural and functional components of the prosthetic valve 1200 can be similar to those described above with respect to any of the prosthetic valves 500, 500, 700, 800, 900, 1000 and 1100 described above, with like numbers referring to like components, and in the interest of brevity will not be further described.

As mentioned above, since a vertical strut 1228, such as a commissure strut 1229 or a non-commissural vertical strut 1230, may not provide sufficient space to include both digits, in some implementations, two adjacent vertical struts 1228, such as a commissure strut 1229 and an adjacent non-commissural vertical strut 1230, or any two adjacent non-commissural vertical struts 1230, can each include one of the digits of a two-digit deployed diameter, which can be similar or different from each other. In the illustrated example, for a prosthetic valve having a deployed diameter of 23 mm, one externally formed information indicator 1250a formed on a non-commissural vertical strut 1230a can take the form of the first digit (e.g., “2”), while another externally formed information indicator 1250b formed on a second non-commissural vertical strut 1230b, adjacent the first non-commissural vertical strut 1230a, can take the form of the second digit (e.g., “3”). In another example (not shown), one externally formed information indicator 1250 formed in a commissure strut 1229 can take the form of the first digit (e.g., “2”), while another externally formed information indicator 1250 formed in a non-commissural vertical strut 1230, adjacent the commissure strut 1229, can take the form of the second digit (e.g., “3”). While prosthetic valve 1200 is described and illustrated to include at least two externally formed information indicators 1250 along two adjacent vertical struts 1228, it is to be understood that any such combination can be implemented for any of the prosthetic valves 500, 500, 700, 800, 900, 1000 and 1100 described above, mutatis mutandis.

While described with respect to a balloon expandable prosthetic valve 1100, it is to be understood that externally formed information indicators of other prosthetic valves disclosed herein, including any of information indicators 550, 750, 850 and 1050 of balloon expandable prosthetic valve 500, 700, 800 and 1000, respectively, as well as information indicator 450 of mechanically expandable prosthetic valve 400, can be similarly designed to include a central portions with a lateral body terminating at a bi-directional vertical extension, mutatis mutandis.

FIG. 6 illustrate a delivery apparatus 600, according to one configuration, adapted to deliver a balloon expandable prosthetic valve 660 described herein (e.g., prosthetic valve 500, 700, 800, 900, 1000 and 1100). It should be understood that the delivery apparatus 600 can be used to implant prosthetic devices other than prosthetic valves, such as stents or grafts.

The delivery apparatus 600 includes a handle 604 and a balloon catheter 652 having an inflatable balloon 650 mounted on its distal end. The balloon expandable prosthetic valve 660 can be carried in a crimped state over the balloon catheter 652. Optionally, an outer delivery shaft 624 can concentrically extend over the balloon catheter 652, and a push shaft 620 disposed over the balloon catheter 652, optionally between the balloon catheter 652 and the outer delivery shaft 624.

The outer delivery shaft 624, the push shaft 620, and the balloon catheter 652, can be configured to be axially movable relative to each other. For example, a proximally oriented movement of the outer delivery shaft 624 relative to the balloon catheter 652, or a distally oriented movement of the balloon catheter 652 relative to the outer delivery shaft 624, can expose the prosthetic valve 660 from the outer delivery shaft 624. The delivery apparatus 600 can further include a nosecone 640 carried by a nosecone shaft (hidden from view in FIG. 6) extending through a lumen of the balloon catheter 652. The nosecone 640 and nosecone shaft can be identical to nosecone 340 and nosecone shaft 332 described for delivery apparatus 300, a detailed description of which is not repeated here in the interest of brevity.

The proximal ends of the balloon catheter 652, the outer delivery shaft 624, the push shaft 620, and optionally the nosecone shaft, can be coupled to the handle 604. During delivery of the prosthetic valve 660, the handle 604 can be maneuvered by an operator (e.g., a clinician or a surgeon) to axially advance or retract components of the delivery apparatus 600, such as the nosecone shaft, the balloon catheter 652, the outer delivery shaft 624, and/or the push shaft 620, through the patient's vasculature, as well as to inflate the balloon 650 mounted on the balloon catheter 652, so as to expand the prosthetic valve 660, and to deflate the balloon 650 and retract the delivery apparatus 600 once the prosthetic valve 660 is mounted in the implantation site.

The handle 604 can include a steering mechanism configured to adjust the curvature of the distal end portion of the delivery apparatus 600. In the illustrated example, the handle 604 includes an adjustment member, such as the illustrated rotatable knob 660a, which in turn is operatively coupled to the proximal end portion of a pull wire. The pull wire can extend distally from the handle 604 through the outer delivery shaft 624 and has a distal end portion affixed to the outer delivery shaft 624 at or near the distal end of the outer delivery shaft 624. Rotating the knob 660a can increase or decrease the tension in the pull wire, thereby adjusting the curvature of the distal end portion of the delivery apparatus 600. Further details on steering or flex mechanisms for the delivery apparatus can be found in U.S. Pat. No. 9,339,384, which is incorporated by reference herein. The handle 604 can further include an adjustment mechanism including an adjustment member, such as the illustrated rotatable knob 606b. The adjustment mechanism can be configured to adjust the axial position of the push shaft 620 relative to the balloon catheter.

The prosthetic valve 660 can be carried by the delivery apparatus 600 during delivery in a crimped state, and expanded by balloon inflation to secure it in a native heart valve annulus. In one exemplary implantation procedure, the prosthetic valve 660 is initially crimped over the balloon catheter 652, proximal to the inflatable balloon 650. Because prosthetic valve 660 is crimped at a location different from the location of balloon 650, prosthetic valve 660 can be crimped to a lower profile than would be possible if it was crimped on top of balloon 650. This lower profile permits the clinician to more easily navigate the delivery apparatus 600 (including crimped prosthetic valve 660) through a patient's vasculature to the treatment location. The lower profile of the crimped prosthetic valve is particularly helpful when navigating through portions of the patient's vasculature which are particularly narrow, such as the iliac artery.

The balloon 650 can be secured to balloon catheter 652 at its balloon proximal end, and to either the balloon catheter 652 or the nosecone 640 at its distal end. The distal end portion of the push shaft 620 is positioned proximal to the outflow end (e.g., outflow end 520, 720, 820, 920, 1020 and 1120) of the prosthetic valve 660.

When reaching the site of implantation, and prior to balloon inflation, the push shaft 620 is advanced distally, allowing its distal end portion to contact and push against the outflow end of prosthetic valve 660, pushing the valve 660 distally therewith. The distal end of push shaft 620 is dimensioned to engage with the outflow end of the prosthetic valve 660 in a crimped configuration of the valve. In some implementations, the distal end portion of the push shaft 620 can be flared radially outward, to terminate at a wider-diameter that can contact the prosthetic valve 660 in its crimped state. Push shaft 620 can then be advanced distally, pushing the prosthetic valve 660 therewith, until the crimped prosthetic valve 660 is disposed around the balloon 650, at which point the balloon 650 can be inflated to radially expand the prosthetic valve 660. Once the prosthetic valve 660 is expanded to its functional diameter within a native annulus, the balloon 650 can be deflated, and the delivery apparatus 600 can be retrieved from the patient's body.

In some examples, the delivery apparatus 300 or 600 with the prosthetic valve 360 or 660, respectively, assembled thereon, can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 360 or 660 and the delivery apparatus 300 or 600, respectively, can be free of any liquid. Methods for treating tissue leaflets for dry storage are disclosed in U.S. Pat. Nos. 8,007,992 and 8,357,387, both of which documents are incorporated herein by reference.

### Some Examples of the Disclosed Technology

Some examples of the above-described technology are enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising:
an annular frame movable between a compressed diameter in a radially compressed state and a deployed diameter in a radially expanded state, the frame comprising:
   a plurality of intersecting angled struts and a plurality of vertical struts, wherein each vertical strut extends between a vertical strut inflow end and an opposite vertical strut outflow end, and wherein the plurality of vertical struts comprises:
      a plurality of commissure struts, each commissure strut comprising a commissure window; and
      a plurality of non-commissural vertical struts, wherein each non-commissural vertical strut is devoid of a commissure window;
   at least one externally formed information indicator formed along at least one of the vertical struts; and
   a valvular structure mounted within the frame and comprising a plurality of leaflets configured to regulate flow through the prosthetic valve;
wherein two of the angled struts intersect with each vertical strut inflow end, and another two of the angled struts intersect with each vertical strut outflow end; and
wherein the width of the externally formed information indicator is at least as great as the width of the vertical strut it is formed along.

Example 2. The prosthetic valve of any example herein, particularly example 1, wherein the externally formed information indicator extends from the vertical strut inflow end of at least one of the commissure struts.

Example 3. The prosthetic valve of any example herein, particularly example 1, wherein the externally formed information indicator extends from the vertical strut outflow end of at least one of the commissure struts.

Example 4. The prosthetic valve of any example herein, particularly example 1, wherein the at least one externally formed information indicator comprises at least two externally formed information indicators, one of which extends from the vertical strut inflow end of at least one of the commissure struts, and the other extends from the vertical strut outflow end of the same commissure strut.

Example 5. The prosthetic valve of any example herein, particularly any one of examples 2 to 4, wherein the at least one externally formed information indicator comprises a plurality externally formed information indicators, formed along a plurality of the commissure struts.

Example 6. The prosthetic valve of any example herein, particularly any one of examples 1 to 5, wherein the externally formed information indicator extends from the vertical strut inflow end of at least one of the non-commissural vertical struts.

Example 7. The prosthetic valve of any example herein, particularly any one of examples 1 to 5, wherein the externally formed information indicator extends from the vertical strut outflow end of at least one of the non-commissural vertical struts.

Example 8. The prosthetic valve of any example herein, particularly any one of examples 1 to 5, wherein the at least one externally formed information indicator comprises at least two externally formed information indicators, one of which extends from the vertical strut inflow end of at least one of the non-commissural vertical struts, and the other extends from the vertical strut outflow end of the same non-commissural vertical strut.

Example 9. The prosthetic valve of any example herein, particularly any one of examples 6 to 8, wherein the at least one externally formed information indicator comprises a plurality externally formed information indicators, formed along a plurality of the non-commissural vertical struts.

Example 10. The prosthetic valve of any example herein, particularly any one of examples 1 to 9, wherein the width of the externally formed information indicator is greater than the width of the vertical strut it is formed along.

Example 11. The prosthetic valve of any example herein, particularly example 7, wherein the width of the externally formed information indicator is greater than the width of the non-commissural vertical strut it is formed on.

Example 12. The prosthetic valve of any example herein, particularly example 11, wherein the difference between the width of the externally formed information indicator and the width of the non-commissural vertical strut is at least as great as the thickness of the leaflet.

Example 13. The prosthetic valve of any example herein, particularly example 11, wherein the difference between the width of the externally formed information indicator and the width of the non-commissural vertical strut is at least as great as twice thickness of the leaflet.

Example 14. The prosthetic valve of any example herein, particularly example 11, wherein the difference between the width of the externally formed information indicator and the width of the non-commissural vertical strut is at least as great as four times the thickness of the leaflet.

Example 15. The prosthetic valve of any example herein, particularly any one of examples 1 to 14, wherein the at least one externally formed information indicator comprises a plurality externally formed information indicators, at least two of which are differently shaped from each other.

Example 16. The prosthetic valve of any example herein, particularly example 15, wherein at least one of the externally formed information indicators is in the shape of a letter, and wherein one other of the externally formed information indicators is in the shape of a digit.

Example 17. The prosthetic valve of any example herein, particularly any one of examples 1 to 17, wherein the frame further comprises at least two support struts extending between at least two adjacent vertical struts and intersecting with each other at a support apex, and wherein at least one support strut extends from the at least one externally formed information indicator.

Example 18. The prosthetic valve of any example herein, particularly example 17, wherein the at least one externally formed information indicator comprises an inflow portion coinciding with the vertical strut inflow end, an opposite outflow portion, and a central portion disposed therebetween, wherein the central portion is in the form of a laterally oriented extension.

Example 19. The prosthetic valve of any example herein, particularly example 18, wherein the support strut extending from the externally formed information indicator, extends from its central portion.

Example 20. The prosthetic valve of any example herein, particularly example 18, wherein the support strut extending from the externally formed information indicator, extends from its outflow portion.

Example 21. The prosthetic valve of any example herein, particularly any one of examples 17 to 20, wherein at least one of the vertical struts from which the support struts extend is a non-commissural vertical strut that further comprises a lateral extension from which the corresponding support strut extends.

Example 22. The prosthetic valve of any example herein, particularly any one of examples 17 to 21, wherein the width of each support strut is less than the width of any of the angled struts.

Example 23. The prosthetic valve of any example herein, particularly any one of examples 17 to 22, wherein the support struts include two support struts extending between each two adjacent vertical struts.

Example 24. The prosthetic valve of any example herein, particularly any one of examples 17 to 23, further comprising an outer skirt disposed around the frame and attached to the support struts.

Example 25. The prosthetic valve of any example herein, particularly any one of examples 1 to 16, wherein the at least one externally formed information indicator comprises an inflow portion connected to two of the angled struts, an opposite outflow portion, and a central portion disposed therebetween, wherein the central portion comprises a lateral body extending in a lateral direction and terminating at a bi-directional vertical extension, forming two shoulders at the transition between the lateral body and the bi-directional vertical extension.

Example 26. The prosthetic valve of any example herein, particularly any one of examples 1 to 16, wherein the at least one externally formed information indicator extends from the vertical strut outflow end of one of the non-commissural vertical strut which is adjacent one of the commissure struts, such that an inflow portion of the externally formed information indicator is axially distal to an outflow edge of the commissure window.

Example 27. The prosthetic valve of any example herein, particularly example 26, wherein an axial distance between the inflow portion of the externally formed information indicator and the outflow edge of the commissure window is less than the difference between an axial height of the prosthetic valve in the radially compressed state and an axial height of the prosthetic valve in the radially expanded state.

Example 28. The prosthetic valve of any example herein, particularly any one of examples 1 to 27, wherein the at least one externally formed information indicator is made of a radiopaque material.

Example 29. The prosthetic valve of any example herein, particularly any one of examples 1 to 28, wherein the at least one externally formed information indicator denotes the manufacturer of the prosthetic valve.

Example 30. The prosthetic valve of any example herein, particularly any one of examples 1 to 29, wherein the at least one externally formed information indicator denotes the model of the prosthetic valve.

Example 31. The prosthetic valve of any example herein, particularly any one of examples 1 to 30, wherein the at least one externally formed information indicator denotes the deployed diameter of the prosthetic valve.

Example 32. The prosthetic valve of any example herein, particularly any one of examples 1 to 30, wherein the at least one externally formed information indicator includes a first externally formed information indicator formed along one of the vertical struts, and a second externally formed information indicator formed along an adjacent vertical strut, wherein the first externally formed information indicator denotes a first digit of the deployed diameter of the prosthetic valve, and the second externally formed information indicator denotes a second digit of the deployed diameter of the prosthetic valve.

Example 33. A prosthetic valve, comprising:
an annular frame movable between a compressed diameter in a radially compressed state and a deployed diameter in a radially expanded state, the frame comprising:
   a plurality of actuation posts comprising upper post members and lower post members;
   a plurality of support posts, wherein each support post extends between a post inflow end and an opposite post outflow end, and wherein the plurality of support posts comprises:
      a plurality of commissure support posts, wherein each commissure support post comprises a commissure window; and
      a plurality of non-commissural support posts, wherein each non-commissural support post is devoid of a commissure window;
a plurality of curved struts extending circumferentially between adjacent actuation posts and support posts and interconnecting the actuation posts and the support posts;
a plurality of actuators coupled to the actuation posts, the actuators configured to adjust the frame between the radially compressed state and the radially expanded state; and
at least one information indicator formed in or along at least one of the support posts; and
a valvular structure mounted within the frame and comprising a plurality of leaflets configured to regulate flow through the prosthetic valve;
wherein two of the curved struts intersect with each post inflow end, and another two of the curved struts intersect with each vertical strut outflow end; and
wherein each support post intersects with at least eight curved struts extending from adjacent actuation posts.

Example 34. The prosthetic valve of any example herein, particularly example 33, wherein the information indicator is an internally formed information indicator.

Example 35. The prosthetic valve of any example herein, particularly example 34, wherein the width of each of the support posts is greater than the width of any of the curved struts.

Example 36. The prosthetic valve of any example herein, particularly example 34 or 35, wherein the width of the support post is greater than the width of the internally formed information indicator formed therein.

Example 37. The prosthetic valve of any example herein, particularly any one of examples 34 to 36, wherein the at least one internally formed information indicator is formed in at least one of the non-commissural support posts.

Example 38. The prosthetic valve of any example herein, particularly any one of examples 34 to 37, wherein the at least one internally formed information indicator comprises at least two internally formed information indicators, both of which are formed at different axial positions of at least one of the non-commissural support posts.

Example 39. The prosthetic valve of any example herein, particularly example 38, wherein at least two of the internally formed information indicators are differently formed.

Example 40. The prosthetic valve of any example herein, particularly any one of examples 34 to 37, wherein the at least one internally formed information indicator comprises a plurality of internally formed information indicators, formed in a plurality of the non-commissural support posts.

Example 41. The prosthetic valve of any example herein, particularly example 40, wherein at least two of the plurality of internally formed information indicators are differently formed.

Example 42. The prosthetic valve of any example herein, particularly any one of examples 34 to 36, wherein the at least one internally formed information indicator is formed in at least one of the commissure support posts.

Example 43. The prosthetic valve of any example herein, particularly example 42, wherein the at least one internally formed information indicator is distal to the commissure support posts.

Example 44. The prosthetic valve of any example herein, particularly example 42 or 43, wherein the at least one internally formed information indicator comprises at least two internally formed information indicators, both of which are formed at different axial positions of at least one of the commissure support posts.

Example 45. The prosthetic valve of any example herein, particularly example 42, wherein at least two of the internally formed information indicators are differently formed.

Example 46. The prosthetic valve of any example herein, particularly any one of examples 42 to 45, wherein the at least one internally formed information indicator comprises a plurality of internally formed information indicators, formed in a plurality of the commissure support posts.

Example 47. The prosthetic valve of any example herein, particularly example 46, wherein at least two of the plurality of internally formed information indicators are differently formed.

Example 48. The prosthetic valve of any example herein, particularly example 33, wherein the information indicator is an externally formed information indicator.

Example 49. The prosthetic valve of any example herein, particularly example 48, wherein the width of the externally formed information indicator is greater than the width of the support post it is formed along.

Example 50. The prosthetic valve of any example herein, particularly example 48 or 49, wherein the width of each of the support posts is greater than the width of any of the curved struts.

Example 51. The prosthetic valve of any example herein, particularly any one of examples 48 to 50, wherein the externally formed information indicator extends from the post inflow end of at least one of the commissure support posts.

Example 52. The prosthetic valve of any example herein, particularly any one of examples 48 to 50, wherein the externally formed information indicator extends from the post outflow end of at least one of the commissure support posts.

Example 53. The prosthetic valve of any example herein, particularly any one of examples 48 to 50, wherein the at least one externally formed information indicator comprises at least two externally formed information indicators, one of which extends from the post inflow end of at least one of the commissure support post, and the other extends from the post outflow end of the same commissure support post.

Example 54. The prosthetic valve of any example herein, particularly any one of examples 51 to 53, wherein the at least one externally formed information indicator comprises a plurality externally formed information indicators, formed along a plurality of the commissure support posts.

Example 55. The prosthetic valve of any example herein, particularly any one of examples 48 to 54, wherein the externally formed information indicator extends from the post inflow end of at least one of the non-commissural support posts.

Example 56. The prosthetic valve of any example herein, particularly any one of examples 48 to 54, wherein the externally formed information indicator extends from the post outflow end of at least one of the non-commissural support posts.

Example 57. The prosthetic valve of any example herein, particularly any one of examples 48 to 54, wherein the at least one externally formed information indicator comprises at least two externally formed information indicators, one of which extends from the post inflow end of at least one of the non-commissural support post, and the other extends from the post outflow end of the same non-commissural support post.

Example 58. The prosthetic valve of any example herein, particularly any one of examples 55 to 57, wherein the at least one externally formed information indicator comprises a plurality externally formed information indicators, formed along a plurality of the non-commissural support posts.

Example 59. The prosthetic valve of any example herein, particularly any one of examples 49 to 58, wherein the at least one externally formed information indicator comprises a plurality externally formed information indicators, at least two of which are differently shaped from each other.

Example 60. The prosthetic valve of any example herein, particularly example 48 or 59, wherein at least one of the externally formed information indicators is in the shape of a letter, and wherein one other of the externally formed information indicators is in the shape of a digit.

Example 61. The prosthetic valve of any example herein, particularly any one of examples 49 to 60, wherein the at least one externally formed information indicator comprises an inflow portion connected to two of the curved struts, an opposite outflow portion, and a central portion disposed therebetween, wherein the central portion comprises a lateral body extending in a lateral direction and terminating at a bi-directional vertical extension, forming two shoulders at the transition between the lateral body and the bi-directional vertical extension.

Example 62. The prosthetic valve of any example herein, particularly any one of examples 49 to 61, wherein the at least one externally formed information indicator is made of a radiopaque material.

Example 63. The prosthetic valve of any example herein, particularly any one of examples 49 to 62, wherein the at least one externally formed information indicator denotes the manufacturer of the prosthetic valve.

Example 64. The prosthetic valve of any example herein, particularly any one of examples 49 to 63, wherein the at least one externally formed information indicator denotes the model of the prosthetic valve.

Example 65. The prosthetic valve of any example herein, particularly any one of examples 49 to 64, wherein the at least one externally formed information indicator denotes the deployed diameter of the prosthetic valve.

Example 66. The prosthetic valve of any example herein, particularly any one of examples 33 to 65, wherein the at least one information indicator includes a first information indicator in one of the support posts, and a second information indicator in an adjacent support post, wherein the first information indicator denotes a first digit of the deployed diameter of the prosthetic valve, and the second information indicator denotes a second digit of the deployed diameter of the prosthetic valve.

It is appreciated that certain features of the invention, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
an annular frame movable between a compressed diameter in a radially compressed state and a deployed diameter in a radially expanded state, the frame comprising:
a plurality of intersecting angled struts and a plurality of vertical struts, wherein each vertical strut extends between a vertical strut inflow end and an opposite vertical strut outflow end, and wherein the plurality of vertical struts comprises:
a plurality of commissure struts, each commissure strut comprising a commissure window; and
a plurality of non-commissural vertical struts;
at least one externally formed information indicator formed along at least one of the vertical struts; and
a valvular structure mounted within the frame and comprising a plurality of leaflets configured to regulate flow through the prosthetic valve.

2. The prosthetic valve of claim 1, wherein the externally formed information indicator extends from the vertical strut inflow end of at least one of the commissure struts.

3. The prosthetic valve of claim 1, wherein the externally formed information indicator extends from the vertical strut outflow end of at least one of the commissure struts.

4. The prosthetic valve of claim 1, wherein the at least one externally formed information indicator comprises at least two externally formed information indicators, one of which extends from the vertical strut inflow end of at least one of the commissure struts, and the other extends from the vertical strut outflow end of the same commissure strut.

5. The prosthetic valve of claim 1, wherein the width of the externally formed information indicator is greater than the width of the vertical strut it is formed along.

6. The prosthetic valve of claim 1, wherein the at least one externally formed information indicator comprises a plurality externally formed information indicators, at least two of which are differently shaped from each other.

7. The prosthetic valve of claim 6, wherein at least one of the externally formed information indicators is in the shape of a letter, and wherein one other of the externally formed information indicators is in the shape of a digit.

8. The prosthetic valve of claim 1, wherein the frame further comprises at least two support struts extending between at least two adjacent vertical struts and intersecting with each other at a support apex, and wherein at least one support strut extends from the at least one externally formed information indicator.

9. The prosthetic valve of claim 8, wherein the at least one externally formed information indicator comprises an inflow portion coinciding with the vertical strut inflow end, an opposite outflow portion, and a central portion disposed therebetween, wherein the central portion is in the form of a laterally oriented extension.

10. The prosthetic valve of claim 1, wherein the at least one externally formed information indicator comprises an inflow portion connected to two of the angled struts, an opposite outflow portion, and a central portion disposed therebetween, wherein the central portion comprises a lateral body extending in a lateral direction and terminating at a bi-directional vertical extension, forming two shoulders at the transition between the lateral body and the bi-directional vertical extension.

11. The prosthetic valve of claim 1, wherein the at least one externally formed information indicator extends from the vertical strut outflow end of one of the non-commissural vertical strut which is adjacent one of the commissure struts, such that an inflow portion of the externally formed information indicator is axially distal to an outflow edge of the commissure window.

12. The prosthetic valve of claim 11, wherein an axial distance between the inflow portion of the externally formed information indicator and the outflow edge of the commissure window is less than the difference between an axial height of the prosthetic valve in the radially compressed state and an axial height of the prosthetic valve in the radially expanded state.

13. The prosthetic valve of claim 1, wherein the at least one externally formed information indicator includes a first externally formed information indicator formed along one of the vertical struts, and a second externally formed information indicator formed along an adjacent vertical strut, wherein the first externally formed information indicator denotes a first digit of the deployed diameter of the prosthetic valve, and the second externally formed information indicator denotes a second digit of the deployed diameter of the prosthetic valve.

14. The prosthetic valve of claim 1, wherein two of the angled struts intersect with each vertical strut inflow end, and another two of the angled struts intersect with each vertical strut outflow end.

15. The prosthetic valve of claim 1, wherein the width of the externally formed information indicator is at least as great as the width of the vertical strut it is formed along.
